(19) United States
(12) Patent Application Publication
(10) Pub. No.: US 2023/0057990 A1
(43) Pub. Date: Feb. 23, 2023

(54) Oral Care Compositions

(71) Applicant: Colgate-Palmolive Company, New York, NY (US)

(72) Inventors: Lauren EVANS, Highland Park, NJ (US); Katarzyna BOCZON, Therwil (CH); Agnieszka KOCINSKA, Basel (CH); Wilbens JOSIAS, North Plainfield, NJ (US); Venda Porter MALONEY, Metuchen, NJ (US); Michael AMOAFO, Bridgewater, NJ (US)

(73) Assignee: Colgate-Palmolive Company, New York, NY (US)

(21) Appl. No.: 17/884,225

(22) Filed: Aug. 9, 2022

Related U.S. Application Data
(60) Provisional application No. 63/231,553, filed on Aug. 10, 2021.

(51) Int. Cl. A61K 8/24; A61K 8/25; A61K 8/27; A61K 8/34; A61Q 11/00

(52) U.S. Cl. CPC A61K 8/24; A61K 8/25; A61K 8/27; A61K 8/345; A61Q 11/00; A61K 2800/30; A61K 2800/48; A61K 2800/42; A61K 2800/412

(57) ABSTRACT

Disclosed herein are oral care compositions that may include, in certain aspects, from about 40 wt. % to about 75 wt. % of a humectant; from about 5 wt. % to about 25 wt. % of an abrasive system; from about 0.3 wt. % to about 1 wt. % of a thickening system; and particles having a refractive index of from about 1.0 to about 2.5; wherein the composition is substantially free of a titanium containing material. Methods of making and using the oral care compositions are also provided.

### CROSS-REFERENCE TO RELATED APPLICATION

This application claims the benefit of priority from U.S. Provisional Application No. 63/231,553, filed Aug. 10, 2021, the contents of which are hereby incorporated herein in their entirety.

### BACKGROUND

Conventional oral care products (e.g., toothpastes) may use titanium dioxide (TiO₂). Utilization of titanium dioxide provides for whitening of the oral care product color, which for many consumers makes it more pleasing to use. Alternatives of TiO₂ may be used, such as a synthetic dye. Unfortunately, compositions having dye may provide for translucent and clear appearance. Moreover, other oral care components, such as sodium fluoride, may be unstable with use of TiO₂ alternatives.

There is a need for oral care compositions that exhibit adequate whiteness and opacity and are stable without the use of with other oral care components.

### BRIEF SUMMARY

This summary is intended merely to introduce a simplified summary of some aspects of one or more implementations of the present disclosure. Further areas of applicability of the present disclosure will become apparent from the detailed description provided hereinafter. This summary is not an extensive overview, nor is it intended to identify key or critical elements of the present teachings, nor to delineate the scope of the disclosure. Rather, its purpose is merely to present one or more concepts in simplified form as a prelude to the detailed description below.

The inventors have discovered that utilization of certain ingredients within a TiO₂ free oral care composition provides for an opaque, white, and stable composition. Furthermore, such composition may be combined with other active ingredients to deliver oral care health benefits in addition to having beneficial aesthetic and stable characteristics.

Thus, in accordance with one aspect, provided is an oral care composition comprising from about 40 wt. % to about 75 wt. % of a humectant; from about 5 wt. % to about 25 wt. % of an abrasive system; from about 0.3 wt. % to about 1 wt. % of a thickening system; and particles having a refractive index from about 1.0 to about 2.5; wherein the composition is substantially free of a titanium containing material. In some embodiments, the humectant is selected from glycerin; sorbitol; and a combination thereof. In some embodiments, the particles are selected from a zinc compound; a calcium compound; a stannous compound; and a combination of two or more thereof. In some embodiments, the particles are selected from zinc oxide; calcium pyrophosphate; dicalcium phosphate dihydrate; calcium carbonate; and stannic oxide. In some embodiments, the particles comprise calcium pyrophosphate. In some embodiments, the particles have a refractive index from about 1.1 to about 2.4. For example, the particles may have a refractive index from about 1.2 to about 2.3, from about 1.3 to about 2.2, from about 1.4 to about 2.1, from about 1.5 to about 2.0, from about 1.5 to about 1.9, from about 1.5 to about 1.8, from about 1.5 to about 1.7, or about 1.5 to about 1.6. In some embodiments, the particles are present in an amount of from about 0.1 wt. % to about 5 wt. %, optionally about 0.25 wt. % to about 4.5 wt. %, or about 0.5 wt. % to about 4 wt. %, or about 1 wt. % to about 3.75 wt. %, or about 1.5 wt. % to about 3.5 wt. %, or about 2 wt. % to about 3.25 wt. %, or about 3 wt. %, based on the total weight of the oral care composition. In some embodiments, the thickening system comprises a thickening agent selected from a carboxyvinyl polymer, carrageenan, xanthan gum, hydroxyethyl cellulose; a water-soluble salt of a cellulose ether (e.g., sodium carboxymethyl cellulose or sodium carboxymethyl hydroxyethyl cellulose); and a combination of two or more thereof. In some embodiments, the composition is free of a titanium containing material. In some embodiments, the titanium containing material comprises titanium dioxide. In some embodiments, the thickening system comprises a thickening agent selected from fumed silica; carboxymethyl cellulose; carboxymethyl hydroxyethyl cellulose; and a combination of two or more thereof. In some embodiments, the thickening system comprises sodium carboxymethyl cellulose. In some embodiments, the abrasive system comprises a precipitated silica, a silica gel and/or high cleaning silica. In some embodiments, the abrasive system comprises a silica having an oil absorption value of less than about 100 cc/100 g silica, less than about 70 cc/100 g silica, or less than about 45 cc/100 g silica. In some embodiments, the abrasive system comprises a silica having an average particle size of from about 3 microns to about 12 microns. In some embodiments, the composition further comprises a surfactant system. In some embodiments, the surfactant system comprises a surfactant selected from an anionic surfactant; a nonionic surfactant; an amphoteric surfactant; and a combination of two or more thereof. In some embodiments, the surfactant system comprises sodium lauryl sulfate and/or cocamidopropyl betaine. In some embodiments, the composition has a Stripe Quality Index (SQI) score of greater than about 1.

In other embodiments, the invention is a method of cleaning teeth, comprising applying an oral care composition according to any embodiment, to an oral cavity surface (e.g., a tooth surface).

In other embodiments, the invention is a method of enhancing the sustainability and/or improving the aesthetic appeal of an oral care composition, comprising admixing an effective amount of particles having a refractive index of from about 1.0 to about 2.5, to an oral composition in need thereof. In certain embodiments, the method further comprises the step of substantially removing all titanium containing materials from the oral care composition.

In other embodiments, the invention is an oral care composition comprising glycerin, sorbitol, or combinations thereof present in an amount from about 40 to about 75 wt. %; water present in an amount from about 5 to about 20 wt. %; silica abrasive present in an amount from about 5 to about 15 wt. % by weight of the composition; a thickening agent present in an amount from about 0.3 to about 1 wt. %; and calcium pyrophosphate present in an amount from about 2 to about 5 wt. %; wherein the composition is substantially free of titanium dioxide, wherein all weight percentages are based on the total weight of the oral care composition. In certain embodiments, the thickening agent is selected from carboxyvinyl polymers, carrageenan, xanthan gum, hydroxyethyl cellulose and water soluble salts of cellulose ethers, such as sodium carboxymethyl cellulose and sodium carboxymethyl hydroxyethyl cellulose. In certain embodiments, the thickening agent is selected from carboxymethyl cellulose and carboxymethyl hydroxyethyl cellulose. In certain embodiments, the thickening agent is sodium carboxymethyl cellulose. In certain embodiments, the silica abrasive is precipitated silica or silica gel. In certain embodiments, the silica abrasive has an oil absorption value of less than 100 cc/100 g silica, less than 70 cc/100 g silica, or less than 45 cc/100 g silica. In certain embodiments, the silica abrasive comprises colloidal particles having an average particle size of from about 3 microns to about 12 microns. In certain embodiments, the silica abrasive comprises colloidal particles having an average particle size of from about 7 microns to about 10 microns. In certain embodiments, the composition further comprises polyethylene glycol. In certain embodiments, the polyethylene glycol is present in an amount from about 1 to about 5 wt. %, by weight of the oral care composition. In certain embodiments, the polyethylene glycol has an average molecular weight of about 200 to about 800. In certain embodiments, the polyethylene glycol has an average molecular weight of about 600. In certain embodiments, the composition further comprises an anionic surfactant. In certain embodiments, the anionic surfactant comprises a water-soluble salt of an alkyl sulfate having from about 10 to about 18 carbon atoms. In certain embodiments, the anionic surfactant comprises sodium lauryl sulfate. In certain embodiments, the anionic surfactant is present in an amount from about 1 to about 3.5 wt. %, by weight of the composition. In certain embodiments, the composition is free of titanium dioxide. In other embodiments, the invention is a method of whitening teeth, comprising applying an oral care composition according to any of the embodiments described herein, to an oral cavity surface (e.g., a surface of a tooth).

In other embodiments, the invention is an oral care composition comprising glycerin, sorbitol, or combinations thereof present in an amount from about 40 to about 75 wt. %; water present in an amount from about 5 to about 20 wt. %; silica abrasive present in an amount from about 5 to about 15 wt. %; a thickening agent present in an amount from about 0.3 to about 1 wt. %,; and tin oxide present in an amount from about 0.5 to about 2 wt. %; wherein the composition is substantially free of titanium dioxide and all weight percentages are based on the total weight of the oral care composition. In certain embodiments, the thickening agent is selected from carboxyvinyl polymers, carrageenan, xanthan gum, hydroxyethyl cellulose and water soluble salts of cellulose ethers, such as sodium carboxymethyl cellulose and sodium carboxymethyl hydroxyethyl cellulose. In certain embodiments, the thickening agent is selected from carboxymethyl cellulose and carboxymethyl hydroxyethyl cellulose. In certain embodiments, the thickening agent comprises sodium carboxymethyl cellulose. In certain embodiments, the silica abrasive comprises precipitated silica or silica gel. In certain embodiments, the silica abrasive has an oil absorption value of less than 100 cc/100 g silica, less than 70 cc/100 g silica, or less than 45 cc/100 g silica. In certain embodiments, the silica abrasive comprises colloidal particles having an average particle size of from about 3 microns to about 12 microns. In certain embodiments, the silica abrasive comprises colloidal particles having an average particle size of from about 7 microns to about 10 microns. In certain embodiments, the composition further comprises polyethylene glycol. In certain embodiments, the polyethylene glycol is present in an amount from about 1 to about 5 wt. %, wherein all weight percentages are based on the total weight of the oral care composition. In certain embodiments, the polyethylene glycol has an average molecular weight of about 200 to about 800. In certain embodiments, the polyethylene glycol has an average molecular weight of about 600. In certain embodiments, the composition further comprises an anionic surfactant. In certain embodiments, the anionic surfactant comprises water-soluble salt of an alkyl sulfate having from about 10 to about 18 carbon atoms. In certain embodiments, the anionic surfactant comprises sodium lauryl sulfate. In certain embodiments, the anionic surfactant is present in an amount from about 1 to about 3.5%, by weight of the composition. In certain embodiments, the composition is free of titanium dioxide. In other aspects, the invention is a method of whitening teeth, comprising applying an oral care composition according to any one of the embodiments described herein to an oral cavity surface (e.g., a surface of a tooth).

Further areas of applicability of the present disclosure will become apparent from the detailed description provided hereinafter. It should be understood that the detailed description and specific examples, while indicating the preferred embodiment of the disclosure, are intended for purposes of illustration only and are not intended to limit the scope of the disclosure.

### BRIEF DESCRIPTION OF THE DRAWINGS

The detailed description of the invention will be better understood when read in conjunction with the appended drawings. It should be understood, however, that the invention is not limited to the precise arrangements and instrumentalities of the embodiments shown in the drawings.

FIG. 1 shows the formulae used to calculate the brightness difference and the hue difference of portions of the oral care striped composition;

FIGS. 2A and 2B show the characterized color breakdown of a first sample of a first control oral care stripe composition;

FIGS. 3A and 3B show the characterized color breakdown of a second sample of a first control oral care stripe composition;

FIGS. 4A and 4B show the characterized color breakdown of a third sample of a first control oral care stripe composition;

FIG. 5 shows a picture of the first comparative oral care stripe composition as well as a width normalized pictograph showing the deviation of the middle color of the first control oral care stripe composition;

FIGS. 6A and 6B show the characterized color breakdown of a first sample of a first exemplary oral care stripe composition in accordance with aspects of the invention;

FIGS. 7A and 7B show the characterized color breakdown of a second sample of the first exemplary oral care stripe composition;

FIGS. 8A and 8B show the characterized color breakdown of a third sample of the first exemplary oral care stripe composition;

FIG. 9 shows a picture of a first sample of the first exemplary composition as well as a width normalized pictograph showing the deviation of the middle color of the first sample of the first exemplary oral care stripe composition;

FIGS. 10A and 10B show the characterized color breakdown of the first sample of the second control oral care stripe composition;

FIGS. 11A and 11B show the characterized color breakdown of a second sample of the second control oral care stripe composition;

FIGS. 12A and 12B show the characterized color breakdown of a third sample of the second control oral care stripe composition;

FIG. 13 shows a picture of a second control oral care stripe composition as well as a width normalized pictograph showing the deviation of the middle color of the second control oral care stripe composition;

FIGS. 14A and 14B show the characterized color breakdown of a first sample of a second exemplary oral care stripe composition;

FIGS. 15A and 15B show the characterized color breakdown of a second sample of the second exemplary oral care stripe composition;

FIGS. 16A and 16B show the characterized color breakdown of a third sample of the second exemplary oral care stripe composition;

FIG. 17 shows a picture of the second exemplary oral care stripe composition as well as a width normalized pictograph showing the deviation of the middle color of the second exemplary oral care stripe composition;

FIGS. 18A and 18B show the characterized color breakdown of a first sample of a third control oral care stripe composition;

FIGS. 19A and 19B show the characterized color breakdown of a second sample of the third control oral care stripe composition;

FIGS. 20A and 20B show the characterized color breakdown of a third sample of the third control oral care stripe composition;

FIG. 21 shows a picture of the third control oral care stripe composition as well as a width normalized pictograph showing the deviation of the middle color of the third control oral care stripe composition;

FIGS. 22A and 22B show the characterized color breakdown of a first sample of a third exemplary oral care stripe composition;

FIGS. 23A and 23B show the characterized color breakdown of a second sample of the third exemplary oral care stripe composition;

FIGS. 24A and 24B show the characterized color breakdown of a third sample of the third exemplary oral care stripe composition; and

FIG. 25 shows a picture of the third exemplary oral care stripe composition as well as a width normalized pictograph showing the deviation of the middle color of the third exemplary oral care stripe composition.

### DETAILED DESCRIPTION

For illustrative purposes, the principles of the present invention are described by referencing various exemplary embodiments thereof. Although certain embodiments of the invention are specifically described herein, one of ordinary skill in the art will readily recognize that the same principles are equally applicable to, and can be employed in other applications and methods. It is to be understood that the invention is not limited in its application to the details of any particular embodiment shown. The terminology used herein is for the purpose of description and not to limit the invention, its application, or uses.

As used herein and in the appended claims, the singular forms “a”, “an”, and “the” include plural references unless the context dictates otherwise. The singular form of any class of the ingredients refers not only to one chemical species within that class, but also to a mixture of those chemical species. The terms “a” (or “an”), “one or more” and “at least one” may be used interchangeably herein. The terms “comprising”, “including”, “containing”, and “having” may be used interchangeably. The term “include” should be interpreted as “include, but are not limited to”. The term “including” should be interpreted as “including, but are not limited to”.

As used throughout, ranges are used as shorthand for describing each and every value that is within the range. Any value within the range can be selected as the terminus of the range. Thus, a range from 1-5, includes specifically 1, 2, 3, 4 and 5, as well as subranges such as 2-5, 3-5, 2-3, 2-4, 1-4, etc. The term “about” when referring to a number means any number within a range of 10% of the number. For example, the phrase “about 2.0 wt. %” refers to a number between and including 1.8 wt. % and 2.2 wt. %. As used herein, the term “substantially free” is intended to mean an amount less than about 5.0 weight %, less than 3.0 weight %, 1.0 wt. %; preferably less than about 0.5 wt. %, and more preferably less than about 0.25 wt. % of the composition. As used herein, the term “free” is intended to mean an amount less than about 0.1 wt. %, and more preferably less than about 0.01 wt. % of the composition.

As used herein, the term “effective amount” refers to an amount that is effective to elicit the desired response, including the amount of a composition that, when administered to a subject, is sufficient to achieve an effect toward the desired result. The effective amount may vary depending on the composition, the disease, and its severity and the age, weight, etc., of the subject to be treated. The effective amount can include a range of amounts. As is understood in the art, an effective amount may be in one or more doses, e.g., a single dose or multiple doses may be required to achieve the desired endpoint.

Unless otherwise specified, all percentages and amounts expressed herein and elsewhere in the specification should be understood to refer to percentages by weight of the total composition. Reference to a molecule, or to molecules, being present at a “wt. %” refers to the amount of that molecule, or molecules, present in the composition based on the total weight of the composition. The symbol “°” refers to a degree, such as a temperature degree or a degree of an angle.

Unless defined otherwise, all technical and scientific terms used herein have the same meanings as commonly understood by one of ordinary skill in the art to which this invention belongs. All patents, patent applications, publications, and other references cited or referred to herein are incorporated by reference in their entireties for all purposes. In the event of a conflict in a definition in the present disclosure and that of a cited reference, the present disclosure controls.

Any member in a list of species that are used to exemplify or define a genus, may be mutually different from, or overlapping with, or a subset of, or equivalent to, or nearly the same as, or identical to, any other member of the list of species. Further, unless explicitly stated, such as when reciting a Markush group, the list of species that define or exemplify the genus is open, and it is given that other species may exist that define or exemplify the genus just as well as, or better than, any other species listed.

All components and elements positively set forth in this disclosure can be negatively excluded from the claims. In other words, the pet food compositions of the instant disclosure can be free or essentially free of all components and elements positively recited throughout the instant disclosure. In some instances, the oral care compositions of the present disclosure may be substantially free of non-incidental amounts of the ingredient(s) or compound(s) described herein. A non-incidental amount of an ingredient or compound is the amount of that ingredient or compound that is added into the oral care composition by itself. For example, an oral care composition may be substantially free of a non-incidental amount of an ingredient or compound, although such ingredient(s) or compound(s) may be present as part of a raw material that is included as a blend of two or more compounds.

Some of the various categories of components identified may overlap. In such cases where overlap may exist and the oral care compositions include both components (or the composition includes more than two components that overlap), an overlapping compound does not represent more than one component. For example, sucralose may be characterized as both a humectant and sweetener. If a particular oral care composition includes sucralose, the sucralose may be characterized only as either a humectant or a sweetener—not both.

Oral care compositions free of titanium dioxide are typically translucent and clear in appearance. Furthermore, oral care compositions having striped, or multi-phase components are generally unstable. The inventors have discovered that the presence of calcium pyrophosphate, tin oxide, or a combination thereof provides for improvement in the opacity and/or whiteness of the composition and stability of the multi-phase components. As such, certain oral care compositions of the invention include calcium pyrophosphate, tin oxide, or a combination thereof to improve the opacity and enhance the whiteness and stability of the oral care composition.

The present disclosure is directed towards oral care whitening compositions comprising oral care composition comprising glycerin, sorbitol, or combinations thereof present in an amount from about 40.0% to about 75.0%, by weight of the composition; water present in an amount from about 5.0% to about 20.0%, by weight of the composition; silica abrasive present in an amount from about 5.0% to about 15.0% by weight of the composition; a thickening agent present in an amount from about 0.3% to about 1.0% by weight of the composition; and an opacifier selected from calcium pyrophosphate present in an amount from about 3% to about 5%, by weight of the composition, tin oxide present in an amount from about 0.5% to about 2.0%, by weight of the composition, or a combination thereof.

The oral care composition of the present invention does require use of titanium dioxide. In certain embodiments, the oral care composition is substantially free of titanium dioxide. In some embodiments, the oral care composition contains less than about 3 wt. % of titanium dioxide, based on the total weight of the oral care composition. For example, the oral care composition may contain less than 0.3 weight %, less than 0.1 weight %, less than 0.01 weight %, or less than 0.001 weight % of titanium dioxide, based on a total weight of the oral care composition. In some embodiments, the oral care composition does not contain titanium dioxide. Additionally or alternatively, the oral care compositions may have less than about 3 wt. % or less of titanium containing ingredients and/or materials, based on the total weight of the oral care composition. For example, oral care compositions may be prepared to have about 3 wt. % or less, about 2 wt. % or less, about 1 wt. % or less, about 0.5 wt. % or less, about 0.1 wt. % or less, about 0.05 wt. % or less, or about 0.01 wt. % or less, of titanium containing ingredients and/or materials. In some embodiments, the oral care composition does not contain titanium containing ingredients and/or materials.

The oral care composition of the present invention may be a multi-phase oral care composition. For example, certain ingredients of the composition may be maintained in one phase, such as a single homogenous aqueous phase, and other ingredients are maintained in another phase, such as a single homogenous non-aqueous phase. In certain aspects, the oral care composition may be a multi-phase oral care composition having two or more stripes. The phases of the oral care compositions may be separate and in contact along an interface. For instance, the plurality of phases may be in the form of stripes with at least two stripes being in contact along an interface. Preferably, when the oral care composition has a plurality of phases in the form of stripes, adjacent stripe contact each other along an interface. For example, the oral care composition may have plurality of phases in the form of stripes, where each stripe contacts each adjacent stripe along an interface. Additionally or alternatively, the two or more phases, e.g., in the form of stripes, may contact each other along a single interface.

The inventors discovered that certain embodiments of the oral care compositions can desirably achieve a stable plurality of phases, such that the interface between each of the phases is substantially uninterrupted between the end points of a sample. For example, the oral care compositions may a plurality of phases in the form of stripes, where the interface defined by the contact of two different phases is uninterrupted for about 80% or more of the length, about 90% or more of the length, about 95% or more of the length, or about 97% or more of the length between the end points of a sample of the oral care composition. The oral care compositions may be prepared with each of the phases individual being aqueous or non-aqueous (e.g., anhydrous). Additionally or alternatively, the phases may individually be a gel or a paste.

The oral care composition of the present invention may comprise calcium pyrophosphate, tin oxide, or a combination thereof. In certain embodiments, the calcium pyrophosphate (Ca₂O₇P₂) is present in an amount from about 2 to about 6 wt. %, from about 2.5 to about 5.5 wt. %, or from about 3 to about 5 wt. %, based on the weight of the composition. For example, the oral are compositions may include calcium pyrophosphate in an amount from about 2 to about 5.5 wt. %, about 2 to about 5 wt. %, about 2 to about 4.5 wt. %, about 2 to about 4 wt. %, about 2 to about 3.5 wt. %, about 2 to about 3 wt. %; from about 2.5 to about 6 wt. %, about 2.5 to about 5.5 wt. %, about 2.5 to about 5 wt. %, about 2.5 to about 4.5 wt. %, about 2.5 to about 4 wt. %, about 2.5 to about 3.5 wt. %, about 2.5 to about 3 wt. %; from about 3 to about 6 wt. %, about 3 to about 5.5 wt. %, about 3 to about 5 wt. %, about 3 to about 4.5 wt. %, about 3 to about 4 wt. %, about 3 to about 3.5 wt. %; from about 3.5 to about 6 wt. %, about 3.5 to about 5.5 wt. %, about 3.5 to about 5 wt. %, about 3.5 to about 4.5 wt. %, about 3.5 to about 4 wt. %; about 4 to about 6 wt. %, about 4 to about 5.5 wt. %, about 4 to about 5 wt. %; from about 4.5 to about 6 wt. %, about 4.5 to about 5.5 wt. %; or from about 5 to about 6 wt. %, including ranges and subranges thereof, based on the total weight of the oral care composition.

In certain embodiments, the oral care compositions comprise a tin oxide in an amount from about 0.5 to about 3 wt. %, from about 0.5 to about 2.5 wt. %, or from about 0.5 to about 2 wt. %, based on the weight of the composition. For example, the oral care compositions may include a tix oxide in amount from about 0.5 to about 2.5 wt. %, about 0.5 to about 2 wt. %, about 0.5 to about 1.5 wt. %, about 0.5 to about 1 wt. %; from about 1 to about 3 wt. %, about 1 to about 2.5 wt. %, about 1 to about 2 wt. %, about 1 to about 1.5 wt. %; from about 1.5 to about 3 wt. %, about 1.5 to about 2.5 wt. %, about 1.5 to about 2 wt. %; from about 2 to about 3 wt. %, about 2 to about 2.5 wt. %; or about 2.5 to about 3 wt. %, including ranges and subranges thereof, based on the total weight of the oral care composition. The tin oxide may be selected from SnO, SnO₂, and a combination thereof.

In certain embodiments, the combination of calcium pyrophosphate and tin oxide is present in a total amount from about 0.5 to about 8 wt. %, from about 0.5 to about 7.5 wt. %, or from about 0.5 to about 5 wt. %, based on the weight of the oral care composition. For example, the oral care compositions may be formulated to include calcium pyrophosphate and tin oxide in a total amount from about 0.5 to about 7 wt. %, about 0.5 to about 6 wt. %, about 0.5 to about 5 wt. %, about 0.5 to about 4 wt. %, about 0.5 to about 3 wt. %, about 0.5 to about 2 wt. %; from about 1 to about 8 wt. %, about 1 to about 7 wt. %, about 1 to about 6 wt. %, about 1 to about 5 wt. %, about 1 to about 4 wt. %, about 1 to about 3 wt. %, about 1 to about 2 wt. %; from about 2 to about 8 wt. %, about 2 to about 7 wt. %, about 2 to about 6 wt. %, about 2 to about 5 wt. %, about 2 to about 4 wt. %, about 2 to about 3 wt. %; from about 3 to about 8 wt. %, about 3 to about 7 wt. %, about 3 to about 6 wt. %, about 3 to about 5 wt. %, about 3 to about 4 wt. %; from about 4 to about 8 wt. %, about 4 to about 7 wt. %, about 4 to about 6 wt. %; from about 5 to about 8 wt. %, about 5 to about 7 wt. %; from about 6 to about 8 wt. %, about 6 to about 7 wt. %, or about 7 to about 8 wt. %, including ranges and subranges thereof, based on the total weight of the oral care composition.

The oral care composition of the present invention may comprise a humectant. Humectants may be useful in order to reduce evaporation and also contribute towards preservation by lowering water activity. Certain humectants can also impart desirable sweetness or flavor to the compositions. Suitable humectants include edible polyhydric alcohols such as glycerin, sorbitol, xylitol, propylene glycol, polyethylene glycol, as well as other polyols and mixtures of these humectants. In certain embodiments, the humectant is sorbitol. In certain embodiments, the sorbitol is in a non-crystal form. In certain embodiments, the humectant is glycerin. In some embodiments, the humectant is a mixture of humectants, such as glycerin and sorbitol, and a polyhydric alcohol, such as propylene glycol, butylene glycol, hexylene glycol, polyethylene glycol. In certain embodiments, mixtures of glycerin and sorbitol may be used as the humectant component of the compositions herein.

The one or more humectants may be present at various amounts or concentrations. In certain embodiments, the one or more humectants are present in an amount from about 40 wt. % to about 75 wt. %, based on the total weight of the oral care composition. For example, humectant may be present in an amount of about 40 wt. %, about 42 wt. %, about 45 wt. %, about 48.5 wt. %, about 53.5 wt. %, about 56.5 wt. %, about 60.5 wt. %, about 62.5 wt. %, about 65 wt. %, about 68 wt. %, about 70 wt. %, about 72 wt. %, about 73.5 wt. %, or about 75 wt. %, including any range formed using the foregoing amounts as end points. For example, humectant(s) may be present in an amount of from about 43 to about 70.0 wt. %, from about 43 to about 68 wt. %, from about 45 to about 70 wt. %, from about 43 to about 68 wt. %, from about 45 to about 60 wt. %, from about 40 to about 60.0 wt. %, or from about 50 to about 70 wt. %, based on the total weight of the oral care composition. In further embodiments, humectant is present in an amount of about 40 wt. % or more, about 43 wt. % or more, about 50 wt. % or more, or about 55 wt. % or more up to about 70 wt. %, based on the weight of the composition. In further embodiments, humectant is present in an amount of about 42.5 to about 72.5 wt. %, about 44 to about 70 wt. %, about 45 to about 68 wt. %, or about 50 to about 70 wt. %, based on the total weight of the oral care composition.

The oral care composition of the present invention comprises thickening agent. Thickening agent(s) provide a desirable consistency or are used to stabilize and/or enhance the solubility of other ingredients. In certain embodiments, the thickening agents are carboxyvinyl polymers, carrageenan, xanthan gum, hydroxyethyl cellulose and water soluble salts of cellulose ethers, such as sodium carboxymethyl cellulose and sodium carboxymethyl hydroxyethyl cellulose. Natural gums such as karaya, gum arabic, and gum tragacanth can also be incorporated. Colloidal magnesium aluminum silicate or finely divided silica can be used as component of the thickening composition to further improve the composition's texture. In some embodiments, thickening agent may include polyethylene glycols, and polysaccharides (e.g., cellulose derivatives, for example carboxymethyl cellulose, or polysaccharide gums, for example xanthan gum or carrageenan gum). In certain embodiments, thickening agents may be selected from carboxyvinyl polymers, carrageenan, xanthan gum, hydroxyethyl cellulose and water soluble salts of cellulose ethers, such as sodium carboxymethyl cellulose and sodium carboxymethyl hydroxyethyl cellulose, and mixtures thereof. In certain embodiments, the acidic polymers, for example carboxymethyl cellulose, may be provided in the form of their free acids or partially or fully neutralized water soluble alkali metal (e.g., potassium and sodium) or ammonium salts. In certain embodiments, the composition comprises a polymer selected from carboxymethyl cellulose (free form or a salt, e.g., sodium salt), a gum (e.g., xanthan gum, carrageenan gum, or gum arabic), polyethylene glycol (e.g., polyethylene glycol 200, 400, 600 or 800), or a mixture thereof. In certain embodiments, the thickening agent is carboxymethylcellulose or a salt derivative thereof. In certain embodiments, the thickening agent is sodium carboxymethylcellulose. In certain embodiments, the oral care composition comprises carboxymethylcellulose and a silica thickener as thickening agents. In certain embodiments, the oral care composition comprises carboxymethylcellulose, polyethylene glycol, and a silica thickener as thickening agents.

The oral care compositions may include a thickening agent that is selected from silica thickeners. Suitable examples of a silica thickener include silica dioxide which corresponds to CAS Registry Number 7631-86-9. Non-limiting examples of silica thickener useful in the practice of the disclosure is Hi-Sil® DT 267-T, by PPG Industries, Inc., Pittsburgh, Pa. or a small particle silica (e.g., Sorbosil AC43 from PQ Corporation, Warrington, United Kingdom). The silica thickener in the oral care composition may, in some cases, be present in an amount of from about 1.5 to about 10 wt. %, about 1.5 to about 8.5 wt. %, about 2 to about 5.5 wt. %, about 2 to about 4 wt. %, or about 2.8 wt. %, by weight of the oral care composition. In certain embodiments, the silica thickener is present in an amount from about 2.5 to about 4 wt. %, by weight of the oral care composition.

The one or more thickening agent may be present at various amounts or concentrations. In certain embodiments, the one or more thickening agent(s) are present in an amount from about 3 to about 10 wt. %, based on the total weight of the oral care composition. For example, thickening agent may be present in an amount of about 3 wt. %, 3.5 wt. %, about 4 wt. %, about 4.5 wt. %, about 5 wt. %, about 5.5 wt. %, about 6 wt. %, about 6.5 wt. %, about 7 wt. %, about 7.5 wt. %, about 8 wt. %, about 8.5 wt. %, about 9 wt. %, about 9.5 wt. %, or about 10 wt. %, including any ranges formed using the foregoing amounts as end points, based on the total weight of the oral care composition. In another example, thickening agent may be present in an amount of from about 3 to about 9.5 wt. %, from about 3 to about 9 wt. %, from about 3.5 to about 9 wt. %, or from about 3.5 to about 8.5 wt. %, based on the total weight of the oral care composition. In further embodiments, thickening agent is present in an amount of about 3 wt. % or more, about 3.3 wt. % or more, about 3.5 wt. % or more up to about 9.0 wt. %, based on the total weight of the oral care composition. In further embodiments, thickening agent is present in an amount of about 3 to about 9.2 wt. %, about 3 to about 9 wt. %, about 3.2 to about 9 wt. %, or about 3.5 to about 8.8 wt. %, including any ranges and subranges thereof, based on the total weight of the oral care composition.

The oral care composition of the present invention may comprise an anticalculus agent. In some instances, the oral care compositions include anticalculus agents in an amount from about 0.1 to about 8 wt. %, based on the total weight of the oral care composition. For example, the anticalculus agent(s) may be present in an amount from about 0.1 to about 7 wt. %, about 0.1 to about 6 wt. %, about 0.1 to about 5 wt. %, about 0.1 to about 4 wt. %, about 0.1 to about 3 wt. %, about 0.1 to about 2 wt. %, about 0.1 to about 1 wt. %; from about 0.5 to about 8 wt. %, about 0.5 to about 7 wt. %, about 0.5 to about 6 wt. %, about 0.5 to about 5 wt. %, about 0.5 to about 4 wt. %, about 0.5 to about 3 wt. %, about 0.5 to about 2 wt. %; from about 1 to about 8 wt. %, about 1 to about 7 wt. %, about 1 to about 6 wt. %, about 1 to about 5 wt. %, about 1 to about 4 wt. %, about 1 to about 3 wt. %, about 1 to about 2 wt. %; from about 2 to about 8 wt. %, about 2 to about 7 wt. %, about 2 to about 6 wt. %, about 2 to about 5 wt. %, about 2 to about 4 wt. %, about 2 to about 3 wt. %; from about 3 to about 8 wt. %, about 3 to about 7 wt. %, about 3 to about 6 wt. %, about 3 to about 5 wt. %, about 3 to about 4 wt. %; from about 4 to about 8 wt. %, about 4 to about 7 wt. %, about 4 to about 6 wt. %; from about 5 to about 8 wt. %, about 5 to about 7 wt. %; from about 6 to about 8 wt. %, about 6 to about 7 wt. %, or about 7 to about 8 wt. %, including ranges and subranges thereof, based on the total weight of the oral care composition. In some embodiments, the anticalculus agent may be present in an amount of from 0.1 to 5 wt. %, e.g., from 0.1 to 3 wt. %, from 0.1 to 2wt. %, from 0.1 to 1 wt. %, from 0.1 to 0.75 wt. %, from 0.2 to 0.75 wt. %, from 0.3 to 0.75 wt. %, or about 0.5 wt. %, based on the total weight of the oral care composition.

Non-limiting illustrative anticalculus agents may include, but are not limited to, phosphates and polyphosphates (for example pyrophosphates and tripolyphosphates), polyaminopropanesulfonic acid (AMPS), hexametaphosphate salts, zinc citrate trihydrate, polypeptides, polyolefin sulfonates, polyolefin phosphates, and diphosphonates. The compositions of the disclosure thus may comprise phosphate salts in addition to the zinc phosphate. In particular embodiments, these salts are alkali phosphate salts, e.g., salts of alkali metal hydroxides or alkaline earth hydroxides, for example, sodium, potassium, or calcium salts. “Phosphate” as used herein encompasses orally acceptable mono- and polyphosphates, for example, P₁₋₆ phosphates, for example monomeric phosphates such as monobasic, dibasic or tribasic phosphate; and dimeric phosphates such as pyrophosphates; and multimeric phosphates, such as tripolyphosphates, tetraphosphates, hexaphosphates and hexametaphosphates (e.g., sodium hexametaphosphate). In particular examples, the selected phosphate is selected from alkali dibasic phosphate and alkali pyrophosphate salts, e.g., selected from sodium phosphate dibasic, potassium phosphate dibasic, dicalcium phosphate dihydrate, calcium pyrophosphate, tetrasodium pyrophosphate, tetrapotassium pyrophosphate, sodium tripolyphosphate, and mixtures of any of two or more of these. In certain embodiments, the anticalculus agent is tetrasodium pyrophosphate. In at least one embodiment the anticalculus agent(s) is selected from phosphates and polyphosphates (e.g., pyrophosphates), polyaminopropanesulfonic acid (AMPS), hexametaphosphate salts, zinc citrate trihydrate, polypeptides, polyolefin sulfonates, polyolefin phosphates, diphosphonates, and phytate acid or its alkaline salt. In some embodiments, the anticalculus agent comprises tetrasodium pyrophosphate (TSPP), sodium tripolyphosphate (STPP), or a combination thereof. In certain embodiments, the anticalculus agent is TSPP.

The oral care composition of the invention may include fluoride. The fluoride may be supplied from one or more fluoride ion sources. The fluoride may be provided as soluble fluoride salts. A wide variety of fluoride ion-yielding materials can be employed as sources of soluble fluoride in the present compositions. Illustrative fluoride ion sources include, but are not limited to, sodium fluoride, stannous fluoride, potassium fluoride, sodium monofluorophosphate, fluorosilicate salts, such as sodium fluorosilicate and ammonium fluorosilicate, amine fluoride, ammonium fluoride, and combinations thereof. In some embodiment, the fluoride ion source is sodium monofluorophosphate or sodium fluoride. Further examples of suitable fluoride ion-yielding materials are found in U.S. Pat. No. 3,535,421, to Briner et al.; U.S. Pat. No. 4,885,155, to Parran, Jr. et al. and U.S. Pat. No. 3,678,154, to Widder et al., each of which are incorporated herein by reference. In certain embodiments the fluoride ion source includes stannous fluoride, sodium fluoride, sodium monofluorophosphate, as well as mixtures thereof. In certain embodiments, the fluoride source is sodium fluoride.

The amount of the fluoride ion source in the oral care composition may vary, but typically is present in an amount from about 0.1 to about 6 wt. %, based on the total weight of the oral care composition. For example, the oral are compositions may include one or more fluoride ion source(s) in an amount from about 0.1 to about 6, about 0.1 to about 5.5 wt. %, about 0.1 to about 5 wt. %, about 0.1 to about 4.5 wt. %, about 0.1 to about 4 wt. %, about 0.1 to about 3.5 wt. %, about 0.1 to about 3 wt. %, about 0.1 to about 2 wt. %, about 0.1 to about 1 wt. %, or about 0.1 to about 0.5 wt. %; from about 1 to about 6, about 1 to about 5.5 wt. %, about 1 to about 5 wt. %, about 1 to about 4.5 wt. %, about 1 to about 4 wt. %, about 1 to about 3.5 wt. %, about 1 to about 3 wt. %; from about 2 to about 6, about 2 to about 5.5 wt. %, about 2 to about 5 wt. %, about 2 to about 4.5 wt. %, about 2 to about 4 wt. %, about 2 to about 3.5 wt. %, or about 2 to about 3 wt. %, including ranges and subranges thereof, based on the total weight of the oral care composition. The amount of the fluoride ion source in the oral care composition may be greater than about 0.1 wt. % and less than 2 wt. %, less than 1 wt. %, less than 0.6 wt. %, less than 0.5 wt. %, or less than 0.4 wt. %. The fluoride ion sources may be present in an amount sufficient to provide a total of about 100 to about 20,000 ppm, about 200 to about 5,000 ppm, or about 500 to about 2,500 ppm fluoride ions. In certain embodiments, the fluoride ion source is sodium fluoride and is present in an amount from about 0.01 to about 1.14%, by weight of the composition, including all values in between.

The oral care composition of the present invention may comprise one or more anionic surfactants. Suitable anionic surfactants include, without limitation, water-soluble salts of C₈₋₂₀ alkyl sulfates, sulfonated monoglycerides of C₈₋₂₀ fatty acids, sarcosinates, taurates and the like. Illustrative examples of these and other classes include sodium lauryl sulfate, sodium lauroyl sarcosinate, sodium lauryl ether sulfate, ammonium lauryl sulfate, ammonium lauryl ether sulfate, sodium cocoyl monoglyceride sulfonate, sodium lauryl sarcosinate, sodium lauryl isoethionate, sodium laureth carboxylase, sodium coconut monoglyceride sulfonates, and sodium dodecyl benzenesulfonate. In some embodiments, the anionic surfactant is sodium lauryl sulfate (SLS). The anionic surfactant, e.g., sodium lauryl sulfate, may be present in an amount of from 0.3 to 2%, by weight, e.g., 0.3-1.5%, 0.5-2.0%, 0.7-1.5%, or about 1.19%, by weight of the composition. For example, the oral care compositions may include an anionic surfactant(s) in amount from about 0.5 to about 2.5 wt. %, about 0.5 to about 2 wt. %, about 0.5 to about 1.5 wt. %, about 0.5 to about 1 wt. %; from about 1 to about 3 wt. %, about 1 to about 2.5 wt. %, about 1 to about 2 wt. %, about 1 to about 1.5 wt. %; from about 1.5 to about 3 wt. %, about 1.5 to about 2.5 wt. %, about 1.5 to about 2 wt. %; from about 2 to about 3 wt. %, about 2 to about 2.5 wt. %; or about 2.5 to about 3 wt. %, including ranges and subranges thereof, based on the total weight of the oral care composition.

Water is present in the oral compositions of the invention. Water, employed in the preparation of commercial oral compositions should be deionized and free of organic impurities. Water commonly makes up the balance of the compositions and includes about 5 to about 20 wt. %, e.g., about 7 to 20 wt. %, by total weight of the oral care composition. In certain embodiments, water may be present in an amount from about 5 to about 20 wt. %, from about 6 to about 17.5 wt. %, about 7 to about 17.5 wt. %, or about 7 to about 11 wt. %, by total weight of the oral care composition. In some instances, the oral care composition is prepared to have water in an amount from about 5 to about 20 wt. %, about 5 to about 17 wt. %, about 5 to about 14 wt. %, about 5 to about 12 wt. %, about 5 to about 10 wt. %, about 5 to about 8 wt. %; from about 7 to about 20 wt. %, about 7 to about 17 wt. %, about 7 to about 14 wt. %, about 7 to about 12 wt. %, about 7 to about 10 wt. %; from about 9 to about 20 wt. %, about 9 to about 17 wt. %, about 9 to about 14 wt. %, about 9 to about 12 wt. %; from about 11 to about 20 wt. %, about 11 to about 17 wt. %, about 11 to about 14 wt. %; from about 14 to about 20 wt. %, about 14 to about 17 wt. %; or from about 17 to about 20 wt. %, including ranges and subranges thereof, based on the total weight of the oral care composition.

This amount of water includes the free water which is added plus that amount which is introduced with other materials such as with sorbitol or silica or any components of the invention. The Karl Fischer method is a one measure of calculating free water.

The oral care composition of the present invention may comprise a peroxide whitening complex which acts as a source of bound hydrogen peroxide. The whitening complex may contain about 10 to about 30 wt. % hydrogen peroxide, based on the weight of the whitening complex, e.g., about 15 about 25 wt. %, about 15 to about 20 wt. %, or about 18 wt. %, based on the total weight of the oral care composition. For example, the oral care compositions may include peroxide in an amount from about 3 to about 30 wt. %, about 3 to about 25 wt. %, about 3 to about 20 wt. %, about 3 to about 15, about 3 to about 10 wt. %, about 3 to about 5 wt. %; from about 7 to about 30 wt. %, about 7 to about 25 wt. %, about 7 to about 20 wt. %, about 7 to about 15, about 7 to about 10 wt. %; from about 10 to about 30 wt. %, about 10 to about 25 wt. %, about 10 to about 20 wt. %, about 10 to about 15 wt. %; from about 15 to about 30 wt. %, about 15 to about 25 wt. %, about 15 to about 20 wt. %; from about 20 to about 30 wt. %, about 20 to about 25 wt. %; or from about 25 to about 30 wt. %, including ranges and subranges thereof, based on the total weight of the oral care composition. In some embodiments, the total amount of peroxide whitening complex provided delivers about 3 about 8 wt. % of hydrogen peroxide based on the weight of the composition, e.g., about 3.5 about 7.5 wt. %, about 3.5 about 5.5 wt. %, or about 4 wt. %, including ranges and subranges thereof, based on the total weight of the oral care composition. In certain embodiments, the peroxide whitening complex may be present in an amount to provide from about 3 to about 8 wt. % of hydrogen peroxide by weight of the composition. In certain embodiments, the peroxide whitening complex is present in an amount to provide about 3.8 to about 4.2 wt. % by weight of the composition. Peroxide may be bound to a polymer such as PVP (polyvinylpyrrolidone). In some embodiments, the peroxide whitening complex is a crosslinked polyvinylpyrrolidone complexed with hydrogen peroxide (PVP-H₂O₂), e.g., Peroxydone™ XL-10 (Ashland Specialty Chemical).

In some embodiment, the oral care composition of the present invention may have a viscosity of about 10,000 CPS to about 700,000 CPS, preferably about 30,000 CPS to about 300,000 CPS, e.g., based on use of a Brookfield viscometer at 1 RPM using spindle #3.

The oral care composition of the present invention may comprise a basic amino acid in free or salt form. The basic amino acids which can be used in the compositions include not only naturally occurring basic amino acids, such as arginine, lysine, and histidine, but also any basic amino acids having a carboxyl group and an amino group in the molecule, which are water-soluble and provide an aqueous solution with a pH of about 7 or greater. Accordingly, basic amino acids include, but are not limited to, arginine, lysine, citrullene, ornithine, creatine, histidine, diaminobutanoic acid, diaminopropionic acid, salts thereof or combinations thereof. In a particular embodiment, the basic amino acids are selected from arginine, lysine, citrullene, and ornithine. The basic amino acids of the oral care composition may generally be present in the L-form or L-configuration. The basic amino acids may be provided as a salt of a di- or tri-peptide including the amino acid. In some embodiments, at least a portion of the basic amino acid present in the oral care composition is in the salt form. In some embodiments, the basic amino acid is arginine, for example, L-arginine, or a salt thereof. Arginine may be provided as free arginine or a salt thereof. For example, Arginine may be provided as arginine phosphate, arginine hydrochloride, arginine sulfate, arginine bicarbonate, or the like, and mixtures or combinations thereof. The basic amino acid may be provided as a solution or a solid. For example, the basic amino acid may be provided as an aqueous solution. In some embodiment, the amino acid includes or is provided by an arginine bicarbonate solution. For example, the amino acid may be provided by an about 40 wt. % solution of the basic amino acid, such as arginine bicarbonate or alternatively called as arginine carbamate. In some embodiments, the basic amino acid is present in an amount of from about 1 to about 15 wt. %, e.g., from about 1 to about 10 wt. %, from about 1 to about 5 wt. %, from about 1 to about 3 wt. %, from about 1 to about 2 wt. %, from about 1.2 to 1.8 wt. %, from about 1.4 to about 1.6 wt. %, or about 1.5 wt. %, by weight of the oral care composition, being calculated as free base form.

The oral care composition of the present invention may comprise a zinc ion source. The zinc ion source may be or include a zinc ion and/or one or more zinc salts. For example, the zinc salts may at least partially dissociate in an aqueous solution to produce zinc ions. Illustrative zinc salts may include, but are not limited to, zinc lactate, zinc oxide, zinc chloride, zinc phosphate, zinc citrate, zinc acetate, zinc borate, zinc butyrate, zinc carbonate, zinc formate, zinc gluconate, zinc glycerate, zinc glycolate, zinc picolinate, zinc proprionate, zinc salicylate, zinc silicate, zinc stearate, zinc tartrate, zinc undecylenate, and mixtures thereof. In some embodiments, the zinc ion source is present in an amount of from about 0.01 to about 5 wt. %, e.g., about 0.1 to about 4 wt. %, or about 1 to about 3 wt. %, by weight of the oral care composition.

The oral care composition of the present invention may include a stannous ion source. The stannous ion source can be a soluble or an insoluble compound of stannous with inorganic or organic counter ions. Examples include the fluoride, chloride, acetate, hexafluorozirconate, sulfate, tartrate, gluconate, citrate, malate, glycinate, pyrophosphate, metaphosphate, oxalate, phosphate, carbonate salts and oxides of stannous. In some embodiments, the stannous ion source is selected from the group consisting of stannous chloride, stannous fluoride, stannous pyrophosphate, stannous formate, stannous acetate, stannous gluconate, stannous lactate, stannous tartrate, stannous oxalate, stannous malonate, stannous citrate, stannous ethylene glycoxide, and mixtures thereof.

The oral care composition of the present invention may include a preservative. Suitable preservatives include, but are not limited to, sodium benzoate, potassium sorbate, methylisothiazolinone, paraben preservatives, for example methyl p-hydroxybenzoate, propyl p-hydroxybenzoate, and mixtures thereof.

The oral care composition of the present invention may include one or more sweeteners safe for oral application. The sweetener may be, for example, saccharin, aspartame, acesulfame, neotame, cyclamate or sucralose; natural high-intensity sweeteners such as thaumatin, stevioside or glycyrrhizin; or such as xylitol, maltitol or mannitol. In certain embodiments, the sweetener is selected from sucralose, saccharin, aspartame, acesulfame, or a combination thereof. In certain embodiments, the sweetener may be utilized in free form or a salt, e.g., sodium salt. In a certain embodiment, the composition comprises a triple sweetener system of sodium saccharin, sucralose and rebaudioside M (Reb M). The one or more sweeteners may be present in an amount of from about 0.001 to about 1 wt. %, about 0.01 to about 0.8 wt. %, about 0.1 to about 0.8 wt. %, or about 0.1 to about 0.75 wt. %, by total weight of the oral care composition. In certain embodiments, the sweetener is saccharin and is present in an amount from about 0.01 to about 0.7 wt. %, by total weight of the oral care composition.

The compositions of the disclosure include silica abrasive. Examples of suitable silica abrasives include standard cleaning silicas, high cleaning silicas or any other suitable abrasive silicas. In certain embodiments, the silica abrasives can be from precipitated silica or silica gels, such as the silica xerogels described in U.S. Pat. No. 3,538,230, to Pader et al. and U.S. Pat. No. 3,862,307, to Digiulio, the disclosures of which are incorporated herein by reference in their entireties. Particular silica xerogels are marketed under the trade name Syloid® by the W. R. Grace & Co., Davison Chemical Division. The precipitated silica materials include those marketed by the J. M. Huber Corp. under the trade name Zeodent®, including the silica carrying the designation Zeodent 115 and 119. These silica abrasives are described in U.S. Pat. No. 4,340,583, to Wason, the disclosure of which is incorporated herein by reference in its entirety. In certain embodiments, abrasive materials useful in the practice of the oral care compositions in accordance with the disclosure include silica gels and precipitated amorphous silica having an oil absorption value of less than 100 cc/100 g silica, such as from 45 cc/100 g to 70 cc/100 g silica. Oil absorption values are measured using the ASTA Rub-Out Method D281. In certain embodiments, the silicas are colloidal particles having an average particle size of from 3 microns to 12 microns, and from 5 to 10 microns. Examples of low oil absorption silica abrasives useful in the practice of the disclosure are marketed under the trade designation Sylodent XWA, by Davison Chemical Division of W.R. Grace & Co., Baltimore, Md. 21203. Sylodent 650 XWA, a silica hydrogel composed of particles of colloidal silica having a water content of 29%, by weight, averaging from 7 to 10 microns in diameter, and an oil absorption of less than 70 cc/100 g of silica is an example of a low oil absorption silica abrasive useful in the practice of the present disclosure. The silica abrasive in the oral care composition may be present in an amount of from about 4.5 to about 15 wt. %, about 5 to about 12.5 wt. %, about 7 to about 11 wt. %, or about 7 to about 10 wt. %, by total weight of the oral care composition. In certain embodiments, the silica abrasive is present in an amount from about 6.5 to about 12 wt. %, by weight of the composition.

In certain embodiments, additional abrasives that can be used in addition to silica abrasives. Non-limiting examples include a calcium phosphate abrasive, e.g., tricalcium phosphate (Ca₃(PO₄)₂), hydroxyapatite (Ca₁₀(PO₄)₆(OH)₂), or dicalcium phosphate dihydrate (CaHPO₄.2H₂O, also sometimes referred to herein as DiCal) or calcium pyrophosphate; calcium carbonate abrasive; or abrasives such as sodium metaphosphate, potassium metaphosphate, aluminum silicate, calcined alumina, bentonite or other siliceous materials, or combinations thereof.

The oral care compositions may include any of the following additional ingredients in an amount of from about 0.01 to about 15 wt. %, based on the total weight of the oral care composition. In some instances, the amount of additional ingredients present in the oral care composition is from about 0.01 to about 12.5 wt. %, about 0.01 to about 10 wt. %, about 0.01 to about 8 wt. %, about 0.01 to about 6 wt. %, about 0.01 to about 4 wt. %, about 0.01 to about 3 wt. %, about 0.01 to about 2 wt. %, about 0.01 to about 1 wt. %, about 0.01 to about 0.5 wt. %, about 0.01 to about 0.1 wt. %; about 0.1 to about 12.5 wt. %, about 0.1 to about 10 wt. %, about 0.1 to about 8 wt. %, about 0.1 to about 6 wt. %, about 0.1 to about 5 wt. %, about 0.1 to about 4 wt. %, about 0.1 to about 3 wt. %, about 0.1 to about 2 wt. %, about 0.1 to about 1 wt. %, about 0.1 to about 0.5 wt. %, about 0.1 to about 0.1 wt. %; about 0.5 to about 12.5 wt. %, about 0.5 to about 10 wt. %, about 0.1 to about 8 wt. %, about 0.5 to about 6 wt. %, about 0.5 to about 5 wt. %, about 0.5 to about 4 wt. %, about 0.5 to about 3 wt. %, about 0.5 to about 2 wt. %, about 0.5 to about 1 wt. %; about 0.75 to about 12.5 wt. %, about 0.75 to about 10 wt. %, about 0.75 to about 8 wt. %, about 0.75 to about 6 wt. %, about 0.75 to about 5 wt. %, about 0.75 to about 4 wt. %, about 0.75 to about 3 wt. %, about 0.75 to about 2 wt. %, about 0.75 to about 1 wt. %; about 1 to about 12.5 wt. %, about 1 to about 10 wt. %, about 1 to about 8 wt. %, about 1 to about 6 wt. %, about 1 to about 5 wt. %, about 1 to about 4 wt. %, about 1 to about 3 wt. %, about 1 to about 2 wt. %; about 2 to about 5 wt. %, about 2 to about 4 wt. %, about 2 to about 3 wt. %; about 3 to about 12.5 wt. %, about 3 to about 10 wt. %, about 3 to about 8 wt. %, about 3 to about 6 wt. %, about 3 to about 5 wt. %, or about 3 to about 4 wt. %, including any range or subrange therebetween, based on the total weight of the oral care composition.

The oral care compositions may include one or more additional ingredients including, e.g., non-hydrogen peroxide whitening agents, nonionic surfactants, amphoteric surfactants, cationic surfactants, stannous salts and/or ions thereof, thickening agents, preservatives, emulsify, colorants, pigments other than calcium pyrophosphate and tin oxides, flavoring agents, sweeteners, or the like.

The oral care composition of the present invention may include one or more flavoring agents. Suitable flavoring agents include, but are not limited to, essential oils and various flavoring aldehydes, esters, alcohols, and similar materials, as well as sweeteners such as sodium saccharin. Examples of the essential oils include oils of spearmint, peppermint, wintergreen, sassafras, clove, sage, eucalyptus, marjoram, cinnamon, lemon, lime, grapefruit, and orange. Also useful are such chemicals as menthol, carvone, and anethole. Flavoring agent is typically incorporated in the oral composition at a concentration of about 0.01% to about 3%, by weight of the composition.

The oral care composition of the invention may include an antioxidant. Any orally acceptable antioxidant may be used, including, but not limited to, butylated hydroxyanisole (BHA), butylated hydroxytoluene (BHT), vitamin A, carotenoids, vitamin E, flavonoids, polyphenols, ascorbic acid, herbal antioxidants, chlorophyll, melatonin, or the like, or combinations and mixtures thereof.

The oral care composition of the invention may include one or more colorants. Colorants among those useful herein include pigments, dyes, lakes and agents imparting a particular luster or reflectivity such as pearling agents. In various embodiments, colorants are operable to provide a white or light-colored coating on a dental surface, to act as an indicator of locations on a dental surface that have been effectively contacted by the composition, and/or to modify appearance, in particular color and/or opacity, of the composition to enhance attractiveness to the consumer. Any orally acceptable colorant can be used, including FD&C dyes and pigments, talc, mica, magnesium carbonate, calcium carbonate, magnesium silicate, magnesium aluminum silicate, silica, zinc oxide, red, yellow, brown and black iron oxides, ferric ammonium ferrocyanide, manganese violet, ultramarine, titaniated mica, bismuth oxychloride, and mixtures thereof. In addition or alternative to the above ranges, the one or more colorants are optionally present in a total amount of about 0.001 to about 20 wt. %, for example about 0.01 to about 10 wt. %, about 0.1 to about 5 wt. %, or about 0.01 to about 1 wt. %, based on the total weight of the oral care composition.

The oral care composition of the invention may include one or more pigments, such as whitening pigments. In some embodiments, the whitening pigments include particles ranging in size from about 0.1 μm to about 10 μm with a refractive index greater than about 1.2. For example, the particles may have an average particle size from about 0.1 to about 10 μm, about 0.1 μm to about 8 μm, about 0.1 μm to about 6 μm, about 0.1 μm to about 4 μm, about 0.1 μm to about 2 μm, about 0.1 μm to about 1 μm; from about 1 to about 10 μm, about 1 μm to about 8 μm, about 1 μm to about 6 μm, about 1 μm to about 4 μm, about 1 μm to about 2 μm; from about 3 to about 10 μm, about 3 μm to about 8 μm, about 3 μm to about 6 μm; from about 5 to about 10 μm, about 5 μm to about 8 μm; from about 7 to about 10 μm, or any ranges or subrange thereof. Additionally or alternatively, the particles may have a refractive index of about 1.2 to about 3, about 1.5 to about 3, about 2 to about 3, about 2.5 to about 3; about 1.2 to about 2.5, about 1.5 to about 2.5, about 2 to about 2.5; from about 2.5 to about 3, or any range or subranges thereof.

Suitable whitening agents include, without limitation, zinc oxide particles, aluminum oxide particles, tin oxide particles, calcium oxide particles, magnesium oxide particles, barium oxide particles, silica particles, zirconium silicate particles, mica particles, talc particles, tetracalcium phosphate particles, amorphous calcium phosphate particles, alpha-tricalcium phosphate particles, beta-tricalcium phosphate particles, hydroxyapatite particles, calcium carbonate particles, zinc phosphate particles, silicon dioxide particles, zirconium silicate particles, or the like, or mixtures and combinations thereof. In certain embodiments, the oral care composition comprises tin oxide, zinc oxide, or a combination thereof.

The compositions and methods according to the invention can be incorporated into oral compositions for the care of the mouth and teeth such as toothpastes, transparent pastes, and gels. In certain embodiments, the oral care composition has an optical density that provides a desirable aesthetic for the oral care composition. In certain embodiments, the optical density provides a clear composition. In other embodiments, the optical density provides for an opaque composition. In further embodiments, the composition has both clear and opaque portions. In certain embodiments, the clear and opaque portions are stripes. In certain embodiments, compositions of the invention have well defined stripe aesthetics. In certain embodiments, the well-defined stripe aesthetics are stable.

All ingredients for use in the compositions described herein should be orally acceptable. As used herein, “orally acceptable” may refer any ingredient that is present in a composition as described in an amount and form which does not render the composition unsafe for use in the oral cavity.

In certain embodiments, the oral care composition is a striped dentifrice comprising a main dentifrice material and a stripe dentifrice material. Striped oral care compositions are known in the art. In certain embodiments, the main dentifrice material is in a different phase as the stripe dentifrice material.

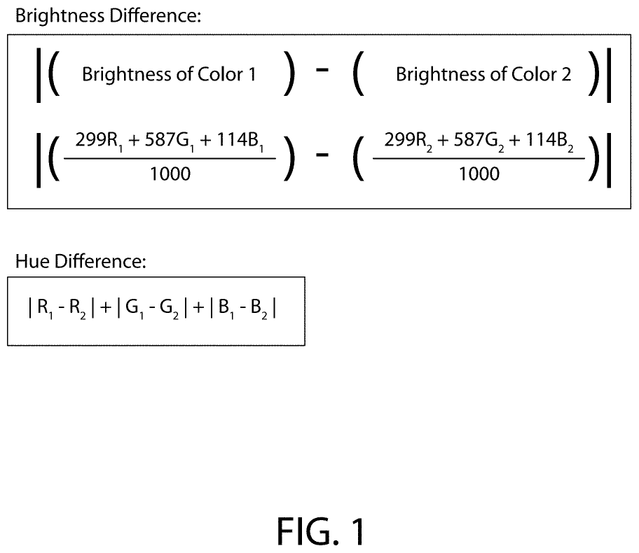

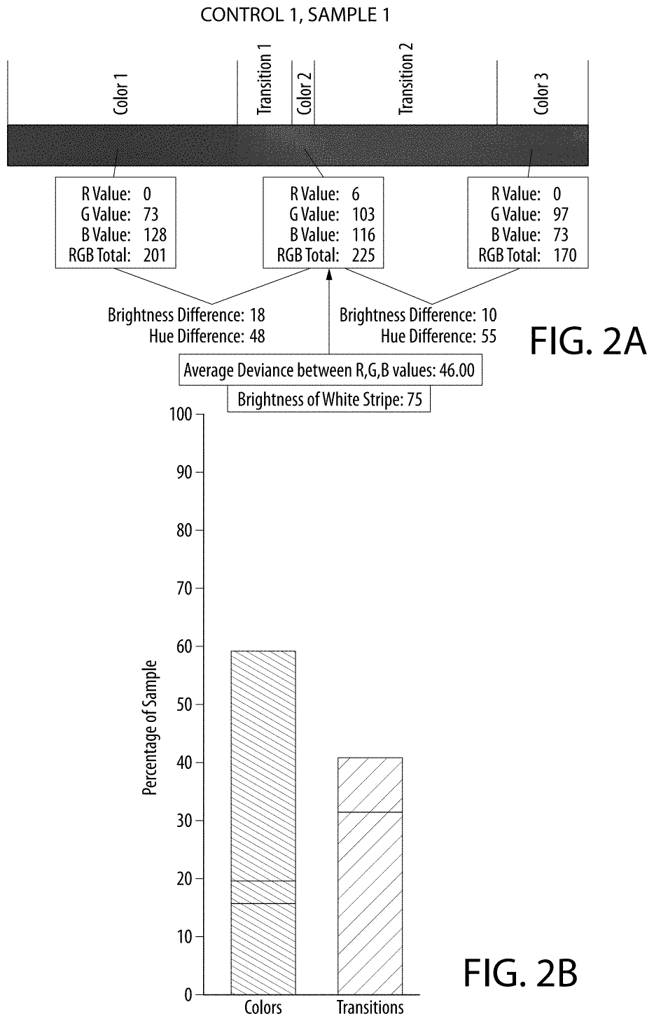

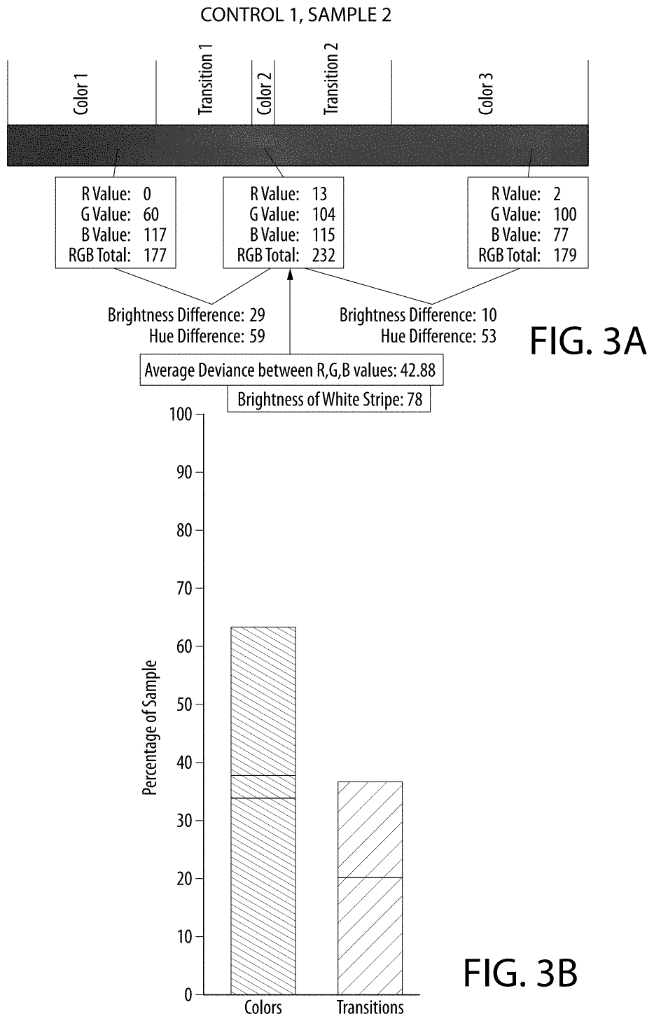

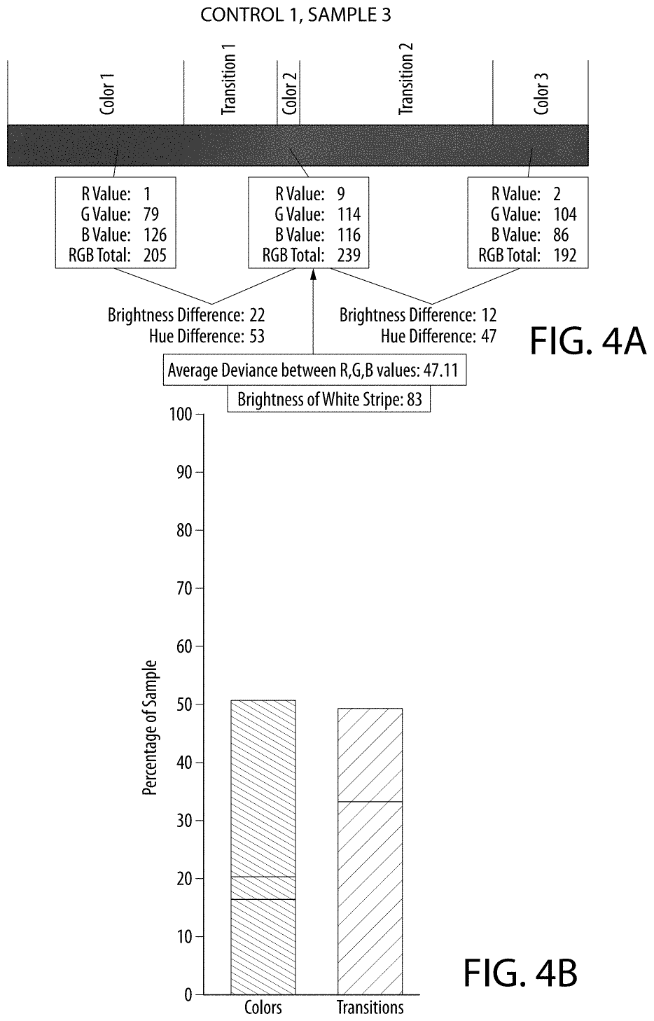

To further characterize oral care compositions of the invention, oral care compositions were photographed and the images digitized and analyzed for various color scheme characteristics. FIG. 1 shows formulae used to calculate the brightness and hue difference. For each composition, three samples of the image were analyzed. FIGS. 2 to 4 show characterization for three samples of control oral care composition, which did not contain calcium pyrophosphate. FIG. 2A shows color values of the composition. FIG. 2B shows the total % contribution from each color and transition of the composition where color 1 corresponds to the bar portion from about 20 to about 60, color 2 corresponds to the bar portion from about 16 to about 20, color 3 corresponds to the bar portion from 0 to about 16, transition 1 corresponds to the bar portion from about 32 to about 40, and transition 2 corresponds to the bar portion from 0 to about 32. FIG. 3A shows color values of the composition. FIG. 3B shows the total % contribution from each color and transition of the composition. FIG. 4A shows color values of the composition. FIG. 4B shows the total % contribution from each color and transition of the composition.

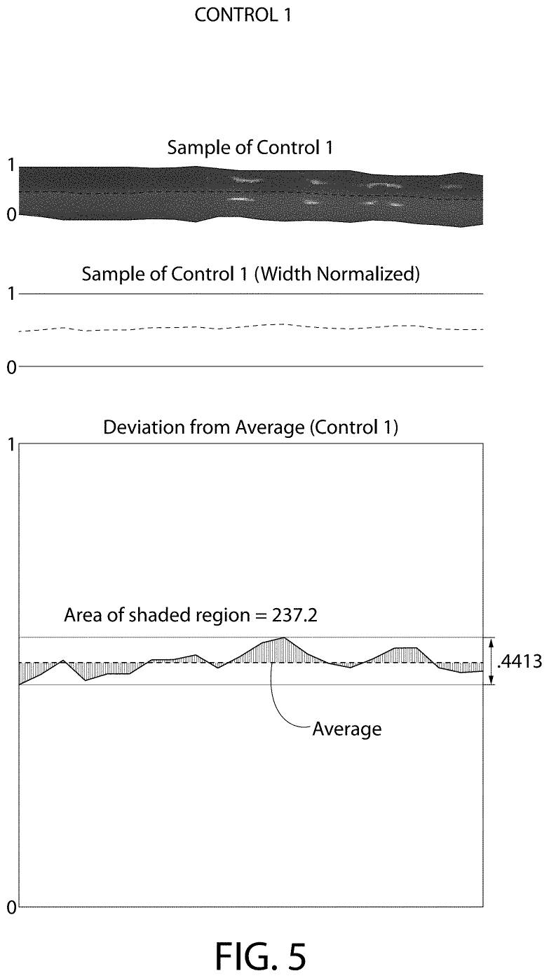

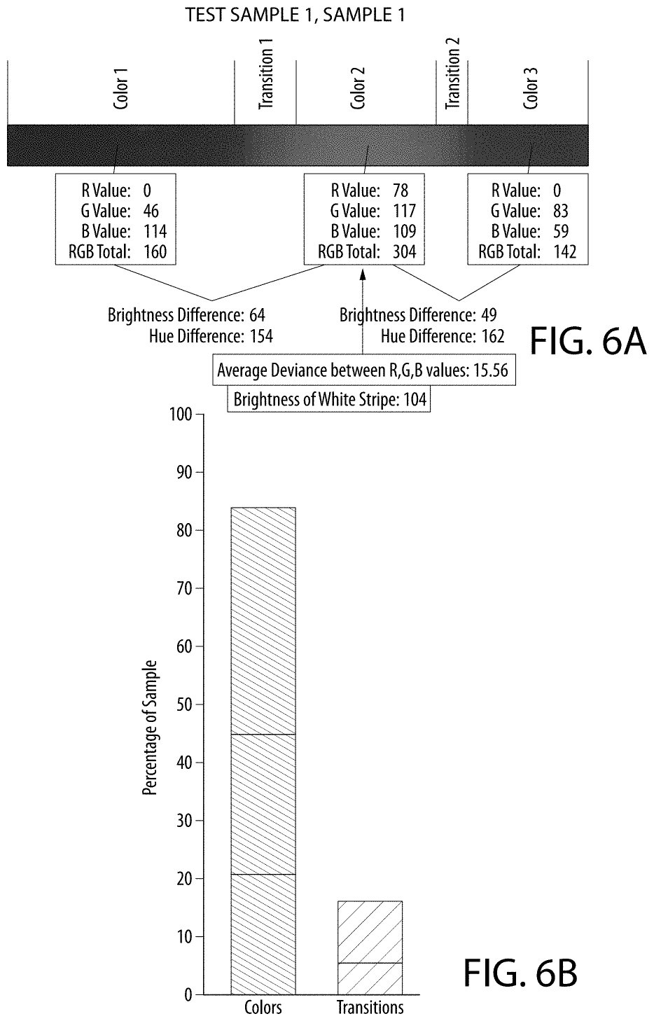

FIG. 5 shows a picture of control 1 oral care stripe composition as well as a width normalized pictograph showing the deviation of the middle color of the first control oral care stripe composition. The deviation relates to an area of deviation of 237.2. FIGS. 6 to 8 show characterization for three samples of a first exemplary oral care composition, which contained calcium pyrophosphate. FIG. 6B shows the total % contribution from each color and transition of the composition where color 1 corresponds to the bar portion from about 45 to about 83, color 2 corresponds to the bar portion from about 20 to about 45, color 3 corresponds to the bar portion from 0 to about 20, transition 1 corresponds to the bar portion from about 5 to about 15, and transition 2 corresponds to the bar portion from 0 to about 5.

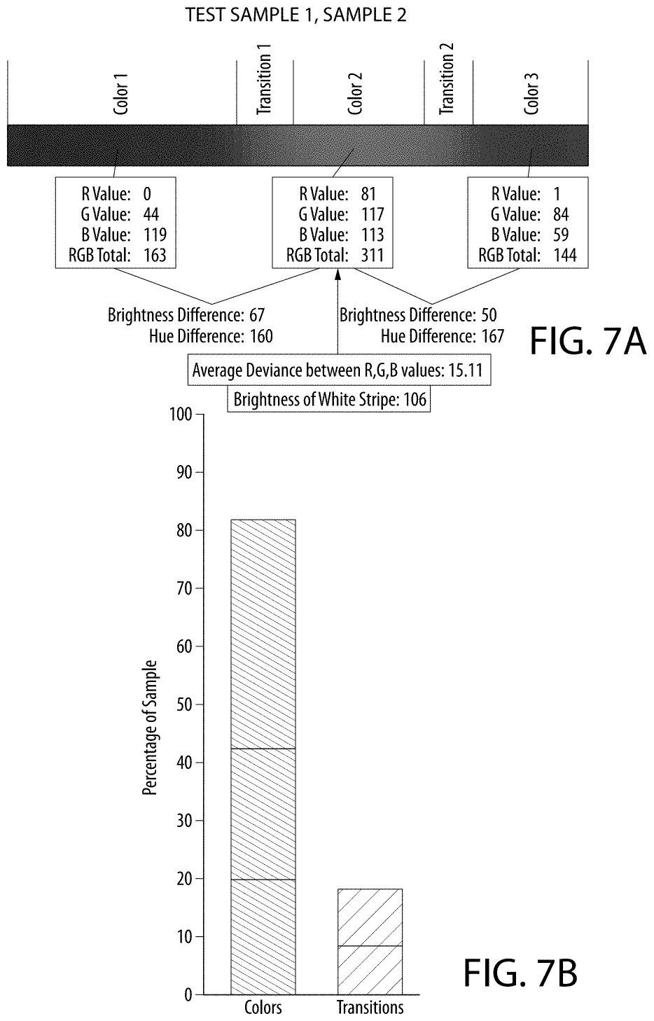

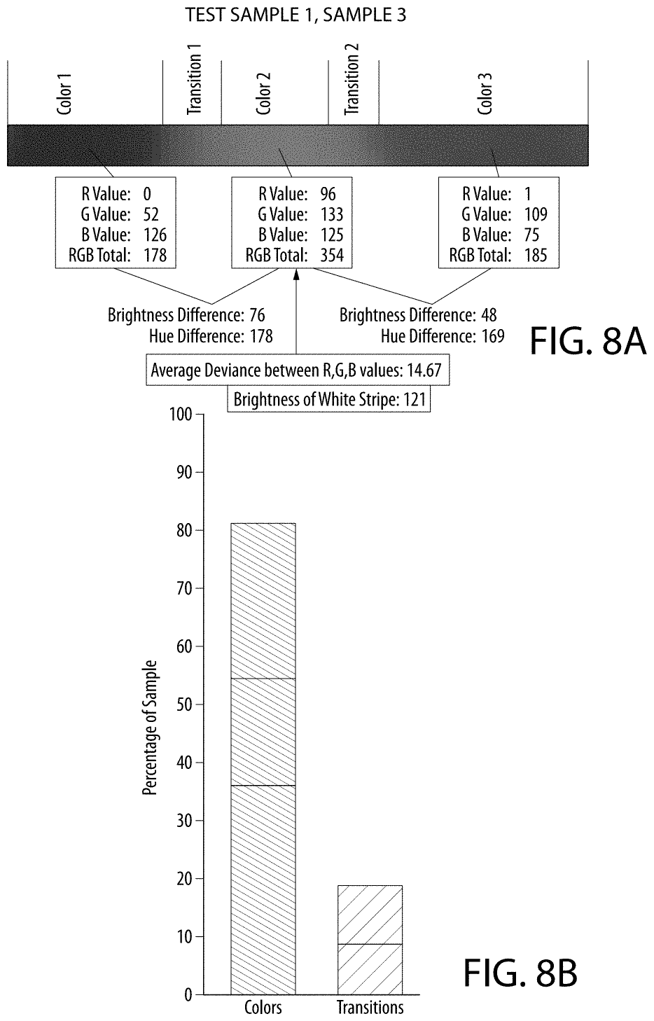

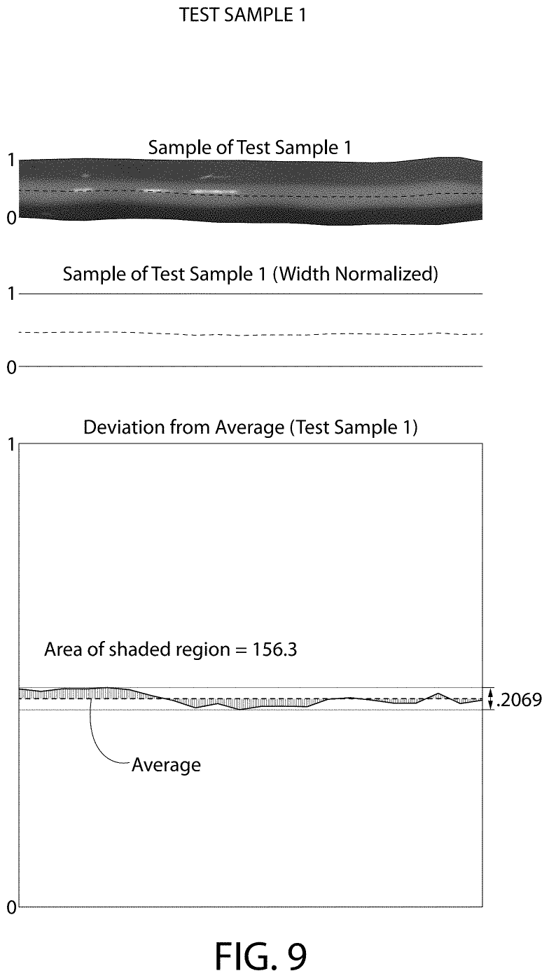

FIG. 7A shows color values of the composition. FIG. 7B shows the total % contribution from each color and transition of the composition. FIG. 8A shows color values of the composition. FIG. 8B shows the total % contribution from each color and transition of the composition. FIG. 9 shows a picture of the first exemplary oral care stripe composition as well as a width normalized pictograph showing the deviation of the middle color of the first exemplary oral care stripe composition. The deviation relates to an area of deviation of 156.3.

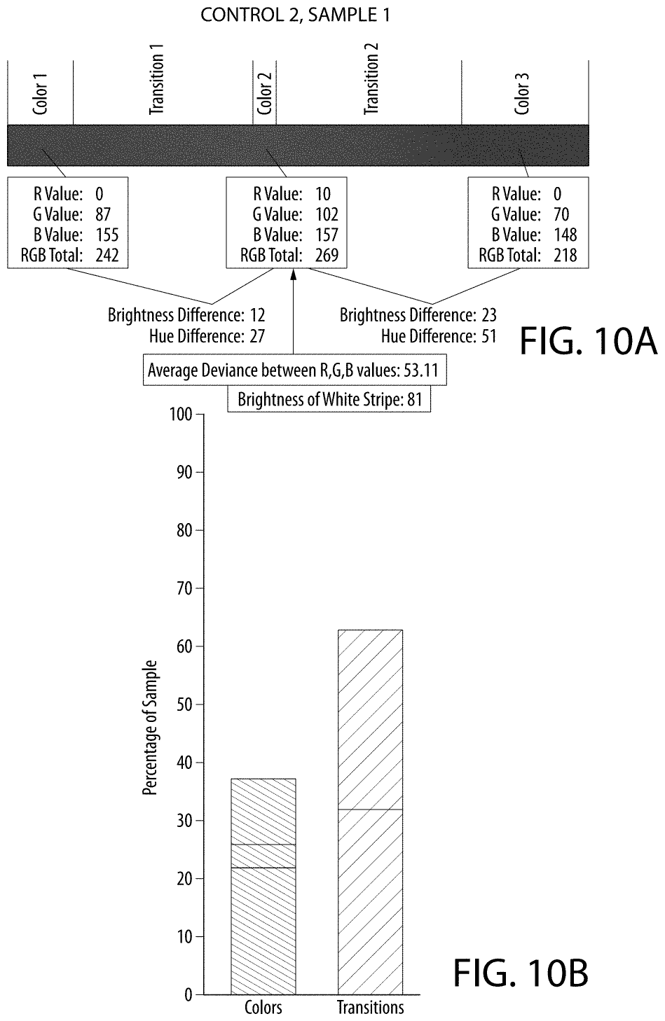

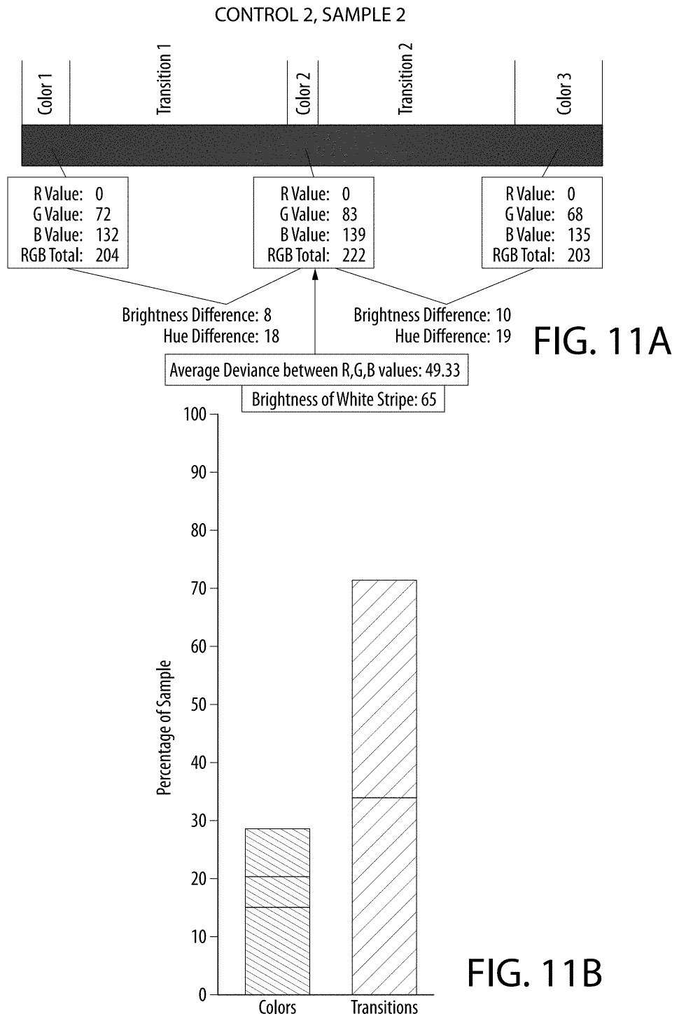

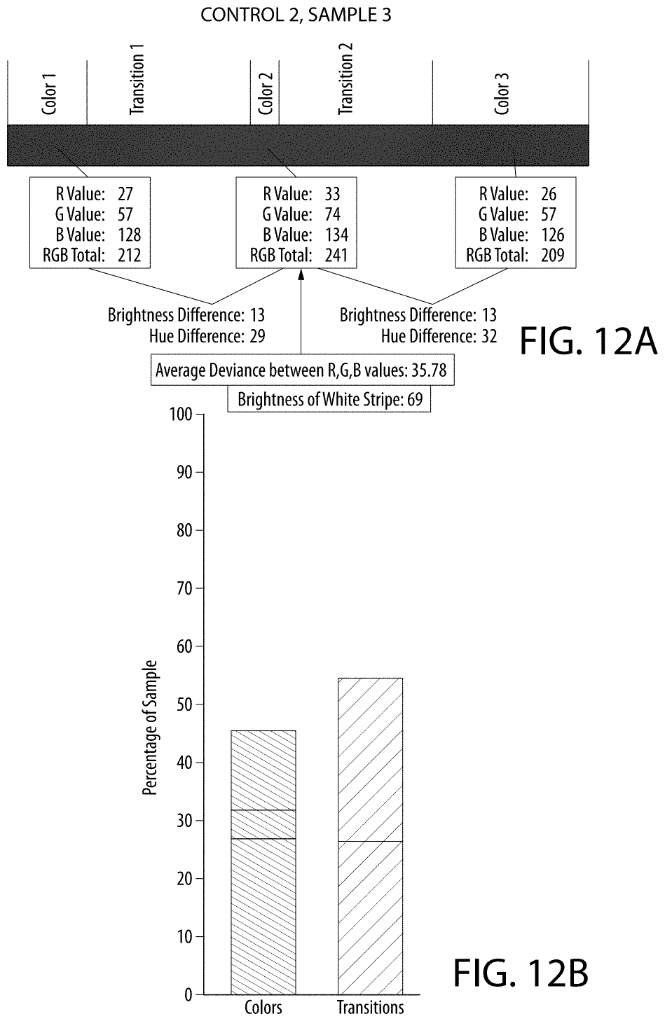

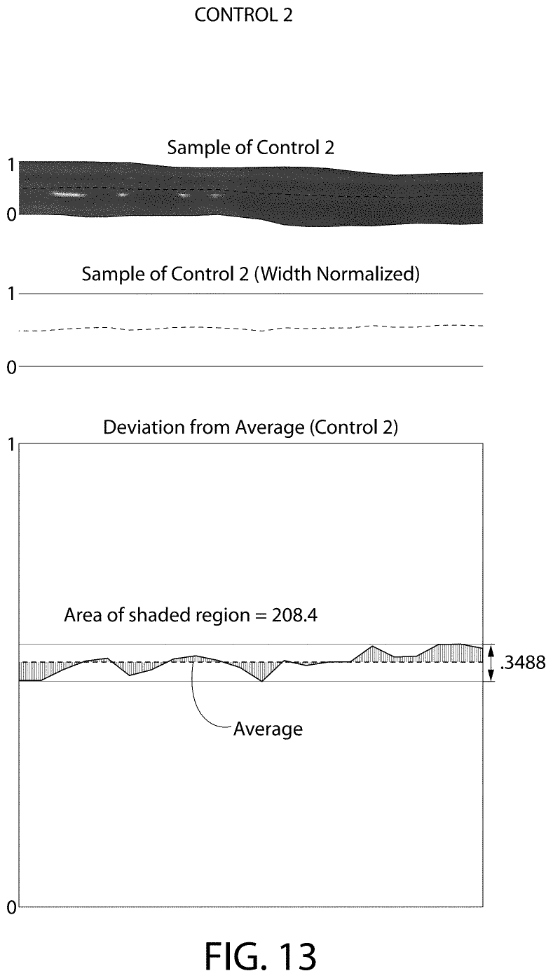

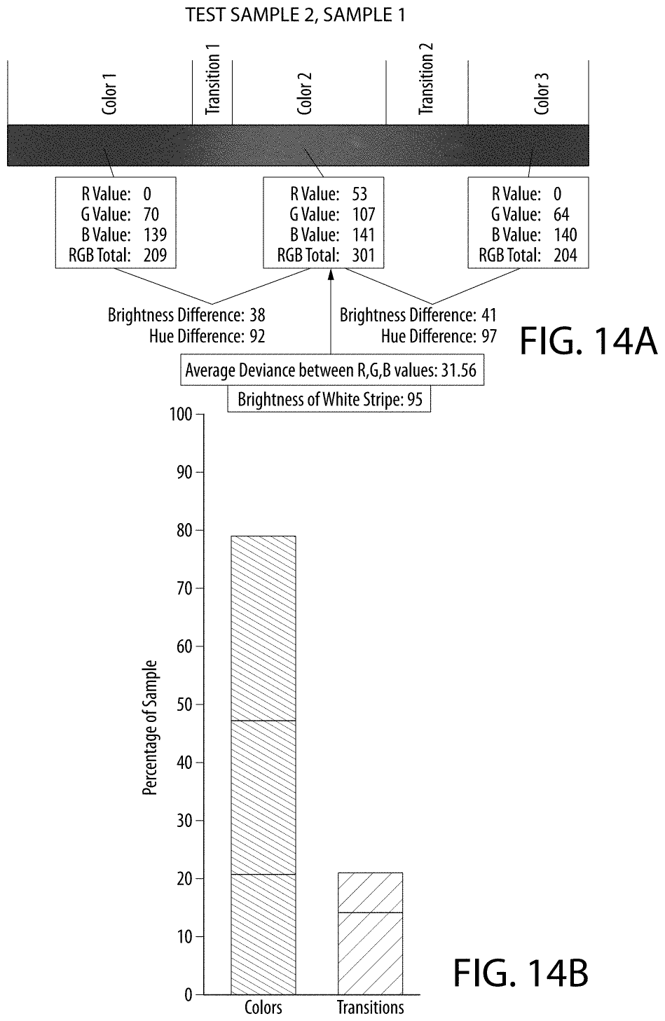

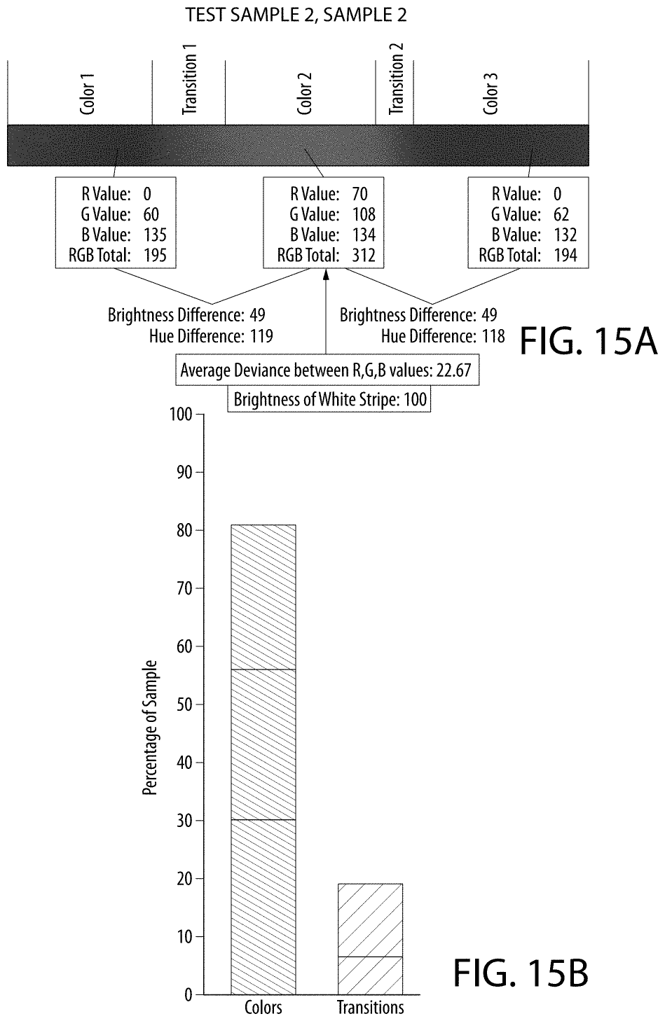

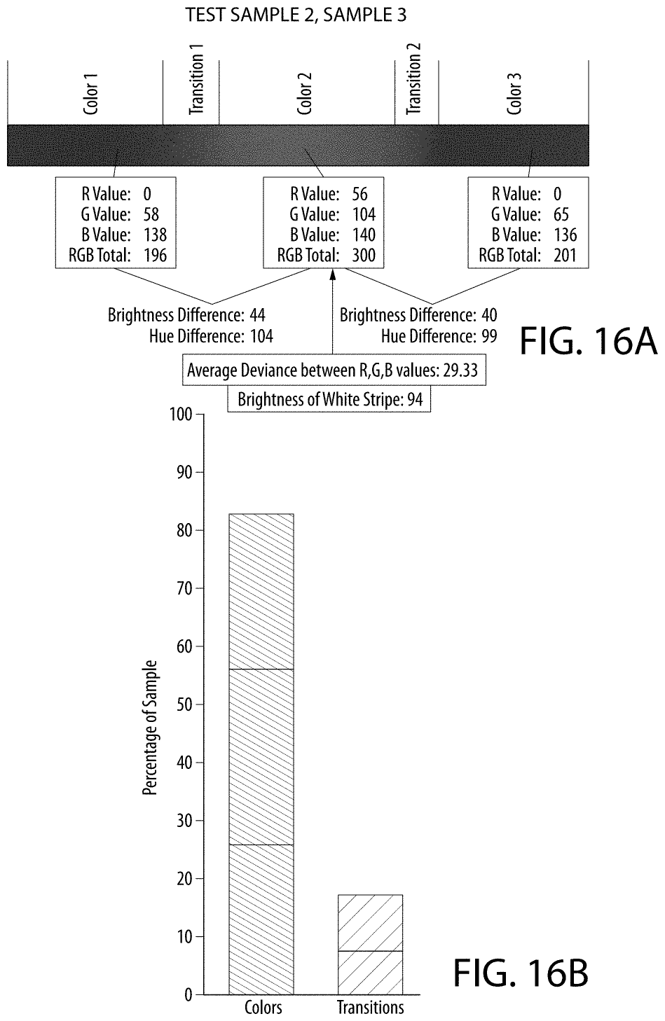

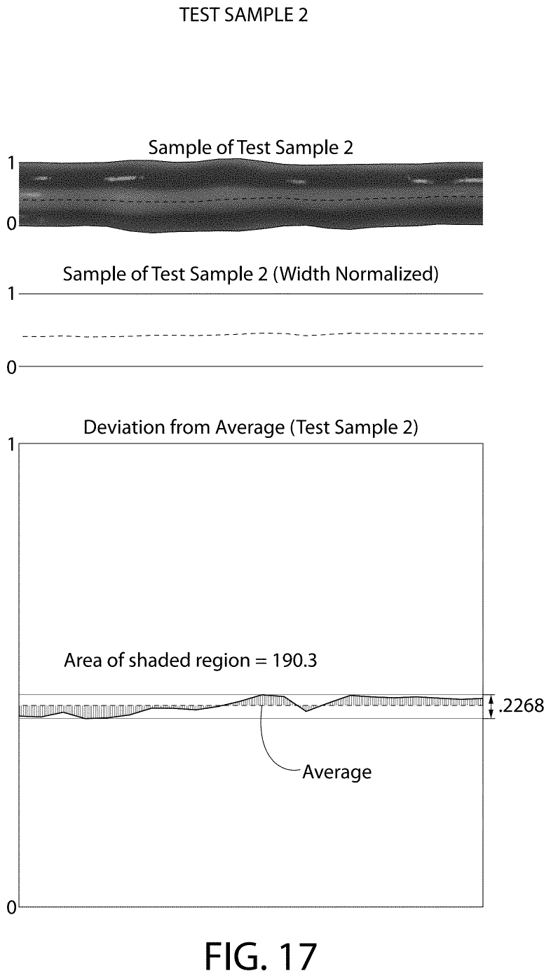

FIGS. 10 to 12 show characterization for three samples of a second control oral care composition, which did not contain calcium pyrophosphate. FIG. 10A shows color values of the composition. FIG. 10B shows the total % contribution from each color and transition of the composition. FIG. 11A shows color values of the composition. FIG. 11B shows the total % contribution from each color and transition of the composition. FIG. 12A shows color values of the composition. FIG. 12B shows the total % contribution from each color and transition of the composition. FIG. 13 shows a picture of the second control oral care stripe composition as well as a width normalized pictograph showing the deviation of the middle color of the second control oral care stripe composition. The deviation relates to an area of deviation of 208.4. FIGS. 14 to 16 show characterization for three samples of a second exemplary oral care composition, which did contain calcium pyrophosphate. FIG. 14A shows color values of the composition. FIG. 14B shows the total % contribution from each color and transition of the composition. FIG. 15A shows color values of the composition. FIG. 15B shows the total % contribution from each color and transition of the composition. FIG. 16A shows color values of the composition. FIG. 16B shows the total % contribution from each color and transition of the composition. FIG. 17 shows a picture of the second exemplary oral care stripe composition as well as a width normalized pictograph showing the deviation of the middle color of the second exemplary oral care stripe composition. The deviation relates to an area of deviation of 190.3.

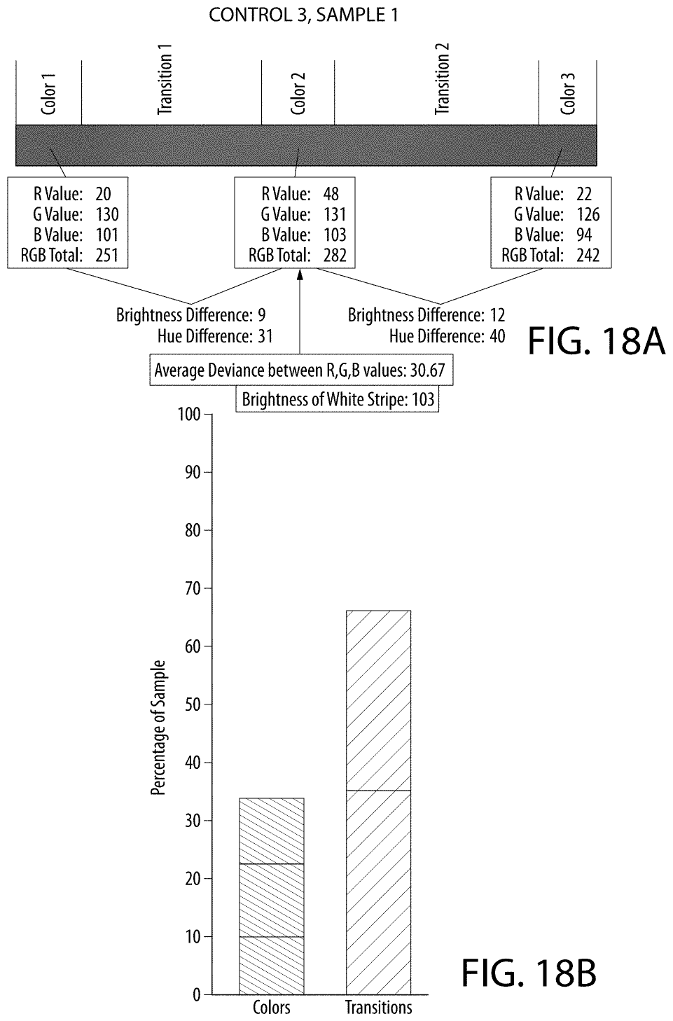

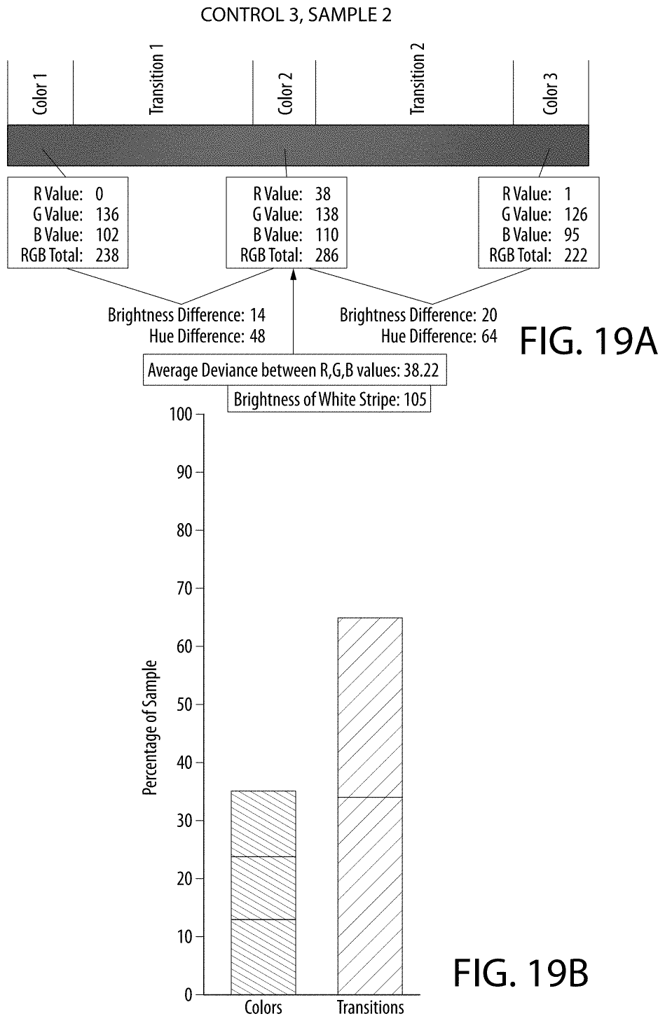

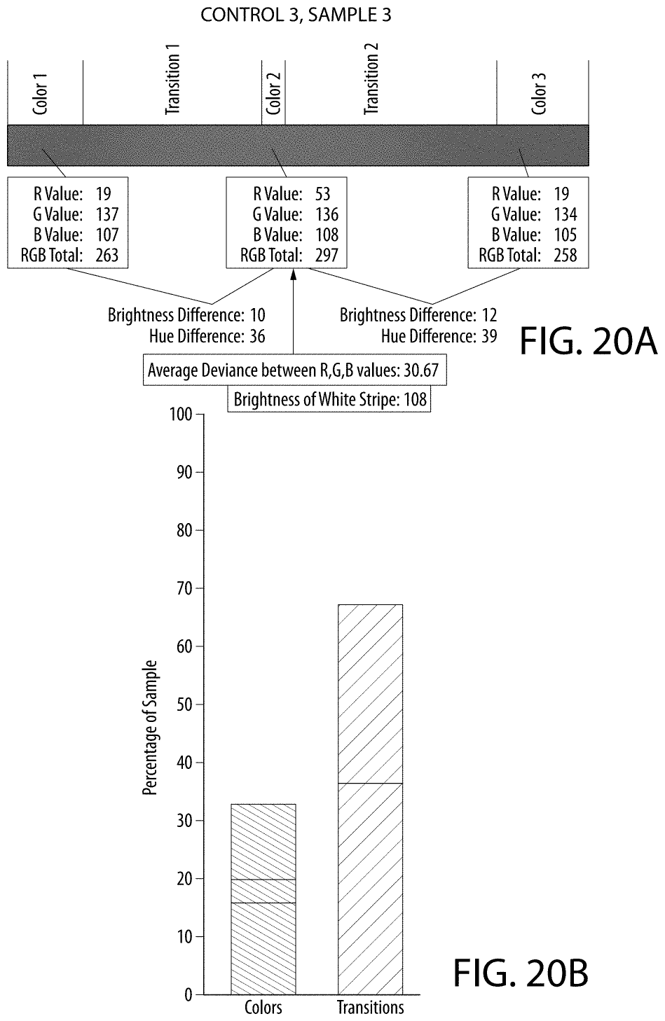

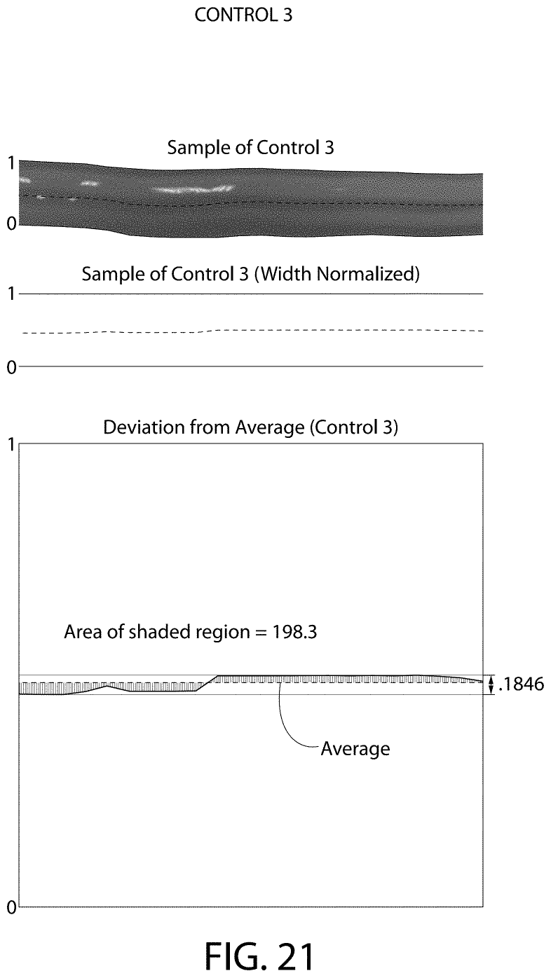

FIGS. 18 to 20 show characterization for three samples of a third control oral care composition, which did not contain calcium pyrophosphate. FIG. 18A shows color values of the composition. FIG. 18B shows the total % contribution from each color and transition of the composition. FIG. 19A shows color values of the composition. FIG. 19B shows the total % contribution from each color and transition of the composition. FIG. 20A shows color values of the composition. FIG. 20B shows the total % contribution from each color and transition of the composition. FIG. 21 shows a picture of control 3 oral care stripe composition as well as a width normalized pictograph showing the deviation of the middle color of the third control oral care stripe composition. The deviation relates to an area of deviation of 198.3.

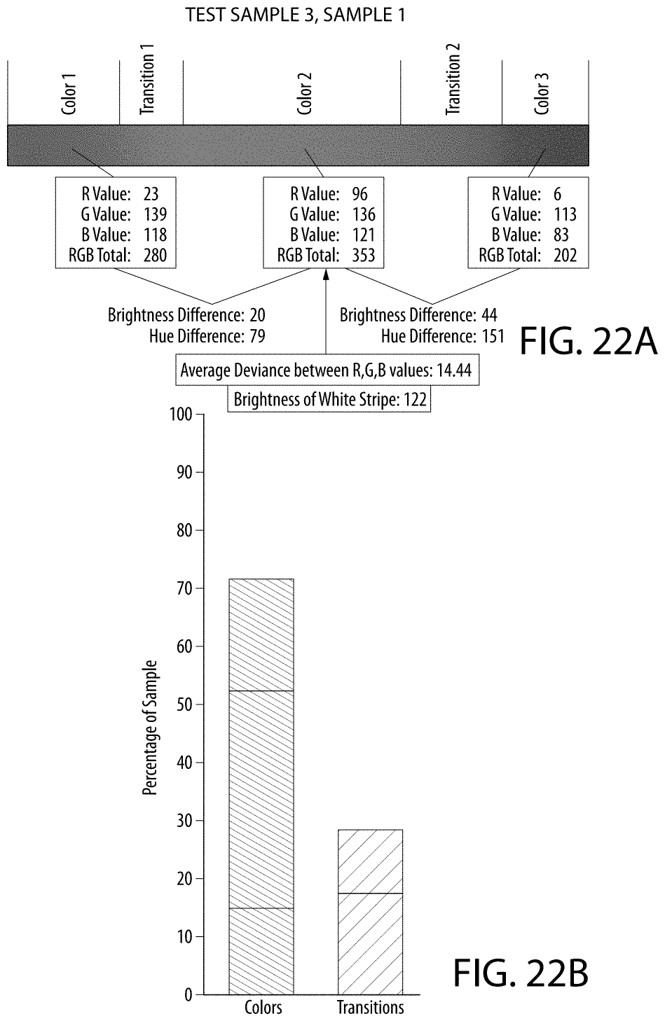

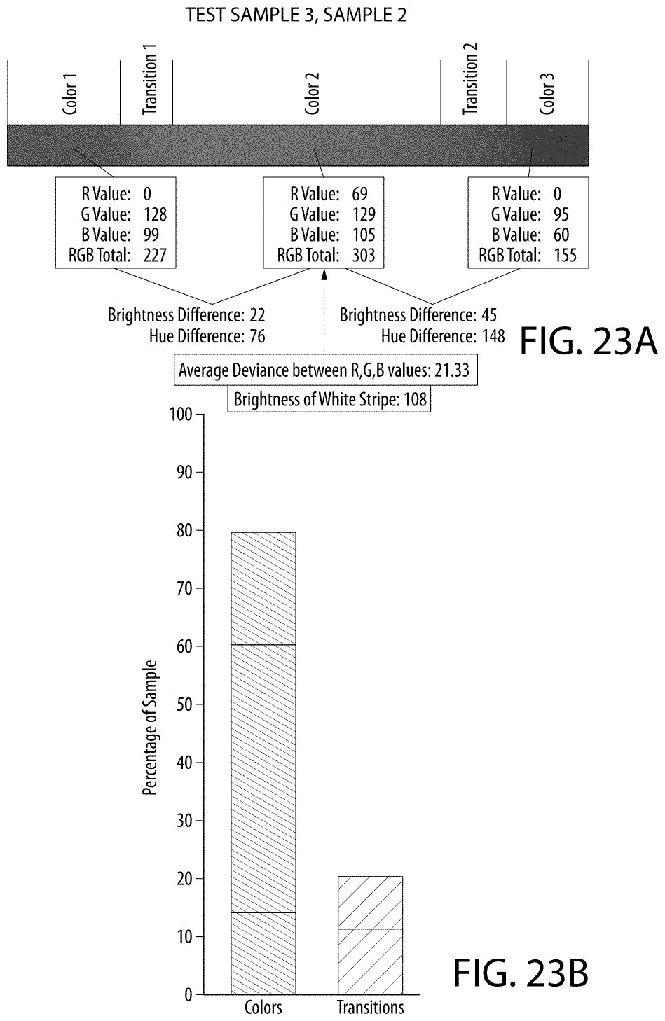

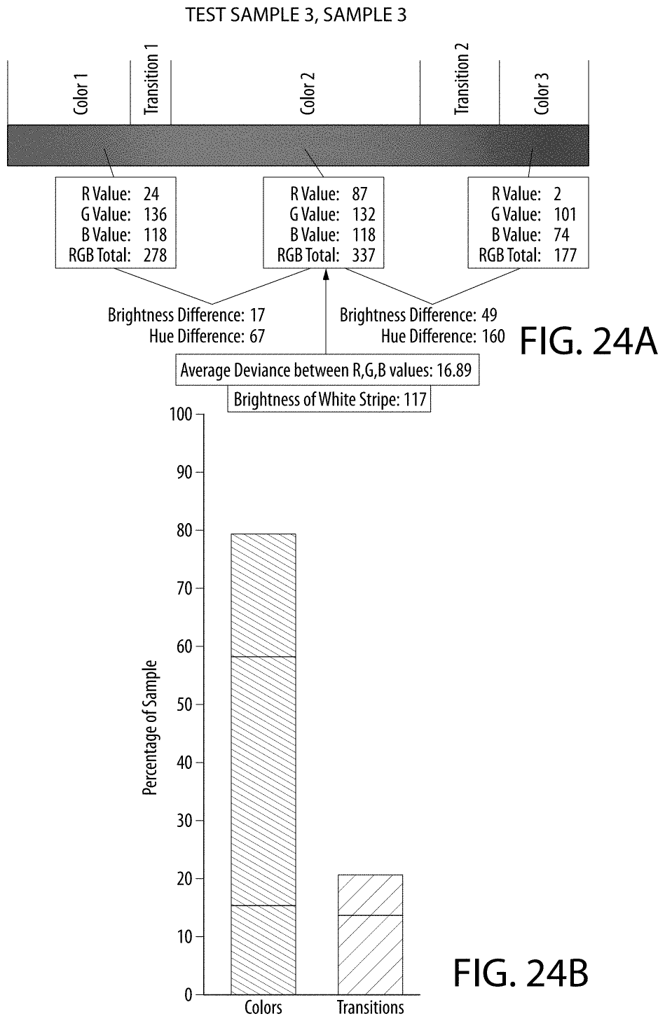

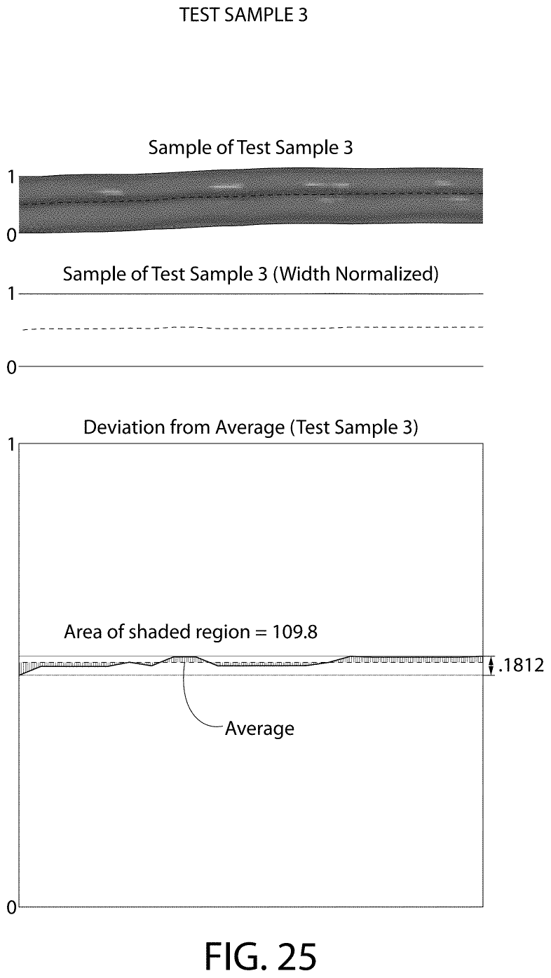

FIGS. 22 to 24 show characterization for three samples of a third exemplary , which contained calcium pyrophosphate. FIG. 22A shows color values of the composition. FIG. 22B shows the total % contribution from each color and transition of the composition. FIG. 23A shows color values of the composition. FIG. 23B shows the total % contribution from each color and transition of the composition. FIG. 24A shows color values of the composition. FIG. 24B shows the total % contribution from each color and transition of the composition. FIG. 25 shows a picture of the third exemplary oral care stripe composition as well as a width normalized pictograph showing the deviation of the middle color of the third exemplary oral care stripe composition. The deviation relates to an area of deviation of 109.8.

Compositions of the invention provided compositions having higher brightness of white stripe, improved difference in hue, as well as superior brightness difference from one color to another. Such aesthetic characteristics provide benefits for both user acceptance and experience.

In another aspect, the present disclosure provides a method of cleaning teeth, the method comprising the application to the oral cavity of a person in need thereof a composition according to the invention (e.g., Composition 1.0 et seq), e.g., by brushing, for example, one or more times per day. In another aspect, the present disclosure provides a method of whitening teeth, the method comprising the application to the oral cavity of a person in need thereof a composition according to the invention (e.g., Composition 1.0 et seq), e.g., by brushing, for example, one or more times per day. In another aspect, the present disclosure provides a method of treatment or prevention of erosive tooth demineralization, gingivitis, plaque, and/or dental caries, the method comprising the application to the oral cavity of a person in need thereof a composition according to the invention (e.g., Composition 1.0 et seq), e.g., by brushing, for example, one or more times per day. In certain aspects, the method comprises applying an effective amount of an oral care composition as described herein to the oral cavity of a person. In another aspect, the present disclosure provides a method of using the compositions described herein (e.g., any of Compositions 1.0 et seq) to treat, reduce or control the incidence of enamel erosion. The methods comprise applying any of the compositions as described herein to the teeth, e.g., by brushing, or otherwise administering the compositions to the oral cavity of a subject in need thereof. The compositions can be administered regularly, such as, for example, one or more times per day. In various embodiments, administering the compositions of the present disclosure to a patient can provide one or more of the following benefits: (i) reduce hypersensitivity of the teeth, (ii) reduce plaque accumulation, (iii) reduce or inhibit demineralization and promote remineralization of the teeth, (iv) inhibit microbial biofilm formation in the oral cavity, (v) reduce or inhibit gingivitis, (vi) promote healing of sores or cuts in the mouth, (vii) reduce levels of acid producing bacteria, (viii) increase relative levels of non-cariogenic and/or non-plaque forming bacteria, (ix) reduce or inhibit formation of dental caries, (x) reduce, repair or inhibit pre-carious lesions of the enamel, e.g., as detected by quantitative light-induced fluorescence (QLF) or electrical caries measurement (ECM), (xi) treat, relieve or reduce dry mouth, (xii) clean the teeth and oral cavity, (xiii) reduce erosion, (xiv) whiten teeth; (xv) reduce tartar build-up, and/or (xvi) promote systemic health, including cardiovascular health, e.g., by reducing potential for systemic infection via the oral tissues.

The disclosure further provides compositions for use in any of the above methods. Further embodiments provide methods wherein at least one tooth is remineralized after administration of a composition as described herein.

The present application further discloses a method of making any of the compositions of the present disclosure. The method comprises combining the cited components in glycerin, sorbitol, or combinations thereof to form a mixture. The amount of glycerin, sorbitol, or combinations thereof employed in the mixture can be any of the amounts recited herein for the compositions of the present disclosure. Any standard mixing techniques can be employed to combine the ingredients and form a stable composition without the need for additional complexing agents.

The present invention, in one of its method aspects, involves applying an oral care composition as described herein to the oral cavity. In certain aspects, the composition is applied at an effective amount.

The invention provides, in an aspect, an oral care composition (Composition 1.0) comprising from about 40 wt. % to about 75 wt. % of a humectant; from about 5 wt. % to about 25 wt. % of an abrasive system; from about 0.3 wt. % to about 1 wt. % of a thickening system; and particles having a refractive index of from about 1.0 to about 2.5; wherein the composition is substantially free of a titanium containing material.

Composition 1.2, wherein the humectant from Composition 1.0 is selected from glycerin; sorbitol; and a combination thereof. Composition 1.3, wherein the particles from Composition 1.0 or 1.2 are selected from a zinc compound; a calcium compound; a stannous compound; and a combination of two or more thereof. Composition 1.4, wherein the particles from any one of compositions of Composition 1.0 to 1.3 are selected from: zinc oxide; calcium pyrophosphate; dicalcium phosphate dihydrate; calcium carbonate; and stannic oxide. Composition 1.5, wherein the particles from any one of compositions of Composition 1.0 to 1.4 comprise calcium pyrophosphate. Composition 1.6, wherein the particles from any one of compositions of Composition 1.0 to 1.5 have a refractive index from about 1.1 to about 2.4, optionally from about 1.2 to about 2.3, from about 1.3 to about 2.2, from about 1.4 to about 2.1, from about 1.5 to about 2.0, from about 1.5 to about 1.9, from about 1.5 to about 1.8, from about 1.5 to about 1.7, or about 1.5 to about 1.6. Composition 1.7, wherein the particles from any one of compositions of Composition 1.0 to 1.6 are present in an amount of from about 0.1 wt. % to about 5 wt. %, optionally about 0.25 wt. % to about 4.5 wt. %, or about 0.5 wt. % to about 4 wt. %, or about 1 wt. % to about 3.75 wt. %, or about 1.5 wt. % to about 3.5 wt. %, or about 2 wt. % to about 3.25 wt. %, or about 3 wt. %, based on the total weight of the oral care composition. Composition 1.8, wherein the thickening system from any one of compositions of Composition 1.0 to 1.7 comprises a thickening agent selected from a carboxyvinyl polymer, carrageenan, xanthan gum, hydroxyethyl cellulose; a water-soluble salt of a cellulose ether (e.g., sodium carboxymethyl cellulose or sodium carboxymethyl hydroxyethyl cellulose); and a combination of two or more thereof. Composition 1.9, wherein the composition from any one of compositions of Composition 1.0 to 1.8 is free of a titanium containing material. Composition 1.10, wherein the titanium containing material from any one of compositions of Composition 1.0 to 1.9 comprises titanium dioxide. Composition 1.11, wherein the thickening system from any one of compositions of Composition 1.0 to 1.10 comprises a thickening agent selected from: fumed silica; carboxymethyl cellulose; carboxymethyl hydroxyethyl cellulose; and a combination of two or more thereof. Composition 1.12, wherein the thickening system from any one of compositions of Composition 1.0 to 1.11 comprises sodium carboxymethyl cellulose. Composition 1.13, wherein the abrasive system from any one of compositions of Composition 1.0 to 1.12 comprises a precipitated silica, a silica gel and/or high cleaning silica. Composition 1.14, wherein the abrasive system from any one of compositions of Composition 1.0 to 1.14 comprises a silica having an oil absorption value of less than about 100 cc/100 g silica, less than about 70 cc/100 g silica, or less than about 45 cc/100 g silica. Composition 1.15, wherein the abrasive system from any one of compositions of Composition 1.0 to 1.14 comprises a silica having an average particle size of from about 3 microns to about 12 microns. Composition 1.16, wherein the composition from any one of compositions of Composition 1.0 to 1.15 further comprises a surfactant system. Composition 1.17, wherein the surfactant system from any one of compositions of Composition 1.0 to 1.16 further comprises a surfactant selected from an anionic surfactant; a nonionic surfactant; an amphoteric surfactant; and a combination of two or more thereof. Composition 1.18, wherein the surfactant system from any one of compositions of Composition 1.0 to 1.17 further comprises sodium lauryl sulfate and/or cocamidopropyl betaine. Composition 1.19, wherein the composition from any one of compositions of Composition 1.0 to 1.18 has an optical density that provides a desirable aesthetic for the oral care composition. In certain embodiments, the desirable aesthetic is for the composition to be clear. In other embodiments, the desirable aesthetic is for the composition to be opaque. Composition 1.20, wherein the composition from any one of compositions of Composition 1.0 to 1.18 has a Stripe Quality Index (SQI) score of greater than about 1. The invention further provides, in an aspect, a method (Method 2.1) of cleaning teeth, comprising applying an oral care composition according to any one of compositions of Composition 1.0 to 1.20, to an oral cavity surface (e.g., a tooth surface).

The invention further provides, in an aspect, a method (Method 2.2) of enhancing the sustainability and/or improving the aesthetic appeal of an oral care composition, comprising admixing an effective amount of particles having a refractive index of from about 1.0 to about 2.5, to an oral composition in need thereof. In certain embodiments, the invention is Method 2.3, wherein the method from Method 2.2 further comprises the step of substantially removing all titanium containing materials from the oral care composition.

The invention provides, in another aspect, an oral care composition (Composition 2.4) comprising glycerin, sorbitol, or combinations thereof present in an amount from about 40.0% to about 75.0%, by weight of the composition; water present in an amount from about 5.0% to about 20.0%, by weight of the composition; silica abrasive present in an amount from about 5.0% to about 15.0% by weight of the composition; a thickening agent present in an amount from about 0.3% to about 1.0% by weight of the composition; and calcium pyrophosphate present in an amount from about 2% to about 5%, by weight of the composition; wherein the composition is substantially free of titanium dioxide.

Composition 2.5, wherein the thickening agent from Composition 2.4 is selected from carboxyvinyl polymers, carrageenan, xanthan gum, hydroxyethyl cellulose and water soluble salts of cellulose ethers, such as sodium carboxymethyl cellulose and sodium carboxymethyl hydroxyethyl cellulose. Composition 2.6, wherein the thickening agent from any one of compositions of Composition 2.4 to 2.5 is selected from carboxymethyl cellulose and carboxymethyl hydroxyethyl cellulose. Composition 2.8, wherein the silica abrasive from any one of compositions of Composition 2.4 to 2.7 is precipitated silica or silica gel. Composition 2.9, wherein the silica abrasive from any one of compositions of Composition 2.4 to 2.8 has an oil absorption value of less than 100 cc/100 g silica, less than 70 cc/100 g silica, or less than 45 cc/100 g silica. Composition 3.0, wherein the silica abrasive from any one of compositions of Composition 2.4 to 2.9 comprises colloidal particles having an average particle size of from about 3 microns to about 12 microns. Composition 3.1, wherein the silica abrasive from any one of compositions of Composition 2.4 to 3.0 comprises colloidal particles having an average particle size of from about 7 microns to about 10 microns. Composition 3.2, wherein the composition from any one of compositions of Composition 2.4 to 3.1 further comprises polyethylene glycol. Composition 3.3, wherein the polyethylene glycol from Composition 3.2 is present in an amount from about 1.0% to about 5.0%, by weight of the composition. Composition 3.4, wherein the polyethylene glycol from any one of compositions of Composition 3.2 to 3.3 has an average molecular weight of about 200 to about 800. Composition 3.5, wherein the polyethylene glycol from any one of compositions of Composition 3.2 to 3.4 has an average molecular weight of about 600. Composition 3.6, wherein the composition from any one of compositions of Composition 2.4 to 3.5 comprises an anionic surfactant. Composition 3.7, wherein the anionic surfactant from Composition 3.6 comprises a water-soluble salt of an alkyl sulfate having from about 10 to about 18 carbon atoms. Composition 3.8, wherein the anionic surfactant from any one of compositions of Composition 3.6 or 3.7 comprises sodium lauryl sulfate. Composition 3.9, wherein the anionic surfactant from any one of compositions of Composition 3.6 to 3.8 is present in an amount from about 1.0% to about 3.5%, by weight of the composition. Composition 4.0, wherein the composition from any one of compositions of Composition 2.4 to 3.9 is free of titanium dioxide. In certain embodiments, the invention is a method of whitening teeth (Method 4.1), wherein the method comprises applying an oral care composition according to any one of compositions 2.4 to 4.0, to an oral cavity surface (e.g., a surface of a tooth). In certain embodiments, the invention is a method of cleaning teeth (Method 6.1), wherein the method comprises applying an oral care composition according to any one of compositions 2.4 to 4.0, to an oral cavity surface (e.g., a surface of a tooth).

The invention provides, in another aspect, an oral care composition (Composition 4.2) comprising glycerin, sorbitol, or combinations thereof present in an amount from about 40.0% to about 75.0%, by weight of the composition; water present in an amount from about 5.0% to about 20.0%, by weight of the composition; silica abrasive present in an amount from about 5.0% to about 15.0% by weight of the composition; a thickening agent present in an amount from about 0.3% to about 1.0% by weight of the composition; and tin oxide present in an amount from about 0.5% to about 2.0%, by weight of the composition; wherein the composition is substantially free of titanium dioxide.

Composition 4.3, wherein the thickening agent from Composition 4.2 is selected from carboxyvinyl polymers, carrageenan, xanthan gum, hydroxyethyl cellulose and water soluble salts of cellulose ethers, such as sodium carboxymethyl cellulose and sodium carboxymethyl hydroxyethyl cellulose. Composition 4.4, wherein the thickening agent from any one of compositions of Composition 4.2 to 4.3 is selected from carboxymethyl cellulose and carboxymethyl hydroxyethyl cellulose. Composition 4.6, wherein the silica abrasive from any one of compositions of Composition 4.2 to 4.4 comprises precipitated silica or silica gel. Composition 4.7, wherein the silica abrasive from any one of compositions of Composition 4.2 to 4.6 has an oil absorption value of less than 100 cc/100 g silica, less than 70 cc/100 g silica, or less than 45 cc/100 g silica. Composition 4.8, wherein the silica abrasive from any one of compositions of Composition 4.2 to 4.7 comprises colloidal particles having an average particle size of from about 3 microns to about 12 microns. Composition 4.9, wherein the silica abrasive from any one of compositions of Composition 4.2 to 4.8 comprises colloidal particles having an average particle size of from about 7 microns to about 10 microns. Composition 5.0, wherein the composition from any one of compositions of Composition 4.2 to 4.9 further comprises polyethylene glycol. Composition 5.1, wherein the polyethylene glycol from Composition 5.0 is present in an amount from about 1.0% to about 5.0%, by weight of the composition. Composition 5.2, wherein the polyethylene glycol from any one of compositions of Composition 5.0 to 5.1 has an average molecular weight of about 200 to about 800. Composition 5.3, wherein the polyethylene glycol from Composition 5.2 has an average molecular weight of about 600. Composition 5.4, wherein the composition from any one of compositions of Composition 4.2 to 5.3 comprises an anionic surfactant. Composition 5.5, wherein the anionic surfactant from Composition 5.4 comprises a water-soluble salt of an alkyl sulfate having from about 10 to about 18 carbon atoms. Composition 5.6, wherein the anionic surfactant from any one of compositions of Composition 5.4 or 5.5 comprises sodium lauryl sulfate. Composition 5.7, wherein the anionic surfactant from any one of compositions of Composition 5.4 to 5.6 is present in an amount from about 1.0% to about 3.5%, by weight of the composition. Composition 5.8, wherein the composition from any one of compositions of Composition 4.2 to 5.7 is free of titanium dioxide. Composition 5.9, wherein the oral care composition from any one of compositions of Composition 1.0 to 1.20, 2.4 to 4.0, or 4.2 to 5.8 comprises tin oxide. Composition 6.0, wherein the oral care composition from any one of compositions of Composition 1.0 to 1.20, 2.4 to 4.0, or 4.2 to 5.9 comprises zinc oxide. In certain embodiments, the invention is a method of whitening teeth (Method 5.9), wherein the method comprises applying an oral care composition according to any one of compositions 4.2 to 5.8, to an oral cavity surface (e.g., a surface of a tooth). In certain embodiments, the invention is a method of cleaning teeth (Method 6.0), wherein the method comprises applying an oral care composition according to any one of compositions 4.2 to 5.8, to an oral cavity surface (e.g., a surface of a tooth).

In certain non-limiting embodiments, the oral care composition may comprise a formulation as specified in the table below.

### EXAMPLES

The examples and other implementations described herein are exemplary and not intended to be limiting in describing the full scope of compositions and methods of this disclosure. The examples disclosed below can further elucidate various benefits and advantages achieved by certain aspects and/or embodiments of the invention. Equivalent changes, modifications and variations of specific implementations, materials, compositions, and methods may be made within the scope of the present disclosure.

### Example 1

A toothpaste composition having the formulation as indicated in Table 1 (below) was prepared.

TABLE 1

| Ingredient | Composition (wt. %) |
|---|---|
| Humectant (e.g., sorbitol) | 60-75 |
| Sodium carboxymethyl cellulose | 0.1-1 |
| Tetrasodium pyrophosphate | 0.1-1 |
| Saccharin | 0.1-0.5 |
| Sodium fluoride | 0.1-0.5 |
| Polyethylene glycol (PEG 600) | 0.1-2 |
| Water | 5-15 |
| Synthetic silica abrasive | 5-15 |
| Synthetic thickening silica | 5-15 |
| Flavor | 0.1-1 |
| 95% sodium lauryl sulfate - granules | 1-2 |
| Blue colorant -1% solution | 0.1-0.5 |
| Total | 100.000 |

### Example 2

A toothpaste composition having the formulation as indicated in Table 2 (below) was prepared.

TABLE 2

| Ingredient | Composition (wt. %) |
|---|---|
| Humectant (e.g., sorbitol) | 50-60 |
| Sodium carboxymethyl cellulose | 0.1-1 |
| Tetrasodium pyrophosphate | 0.1-1 |
| Saccharin | 0.1-1 |
| Sodium fluoride | 0.1-0.5 |
| Polyethylene glycol (peg 600) | 1-5 |
| Water | 5-10 |
| Synthetic high cleaning silica | 5-15 |
| Synthetic silica abrasive | 5-15 |
| Synthetic thickening silica | 1-5 |
| Flavor | 1-5 |
| 28% Sodium lauryl sulfate | 2-8 |
| Dye | 0.0001-0.001 |
| Cocamidopropyl betaine | 1-2 |
| Film (canola free) | 0.1-1 |
| Vegetable carbon | 0.001-0.01 |
| Total | 100.000 |

### Example 3

A toothpaste composition having the formulation as indicated in Table 3 (below) was prepared.

TABLE 3

| Ingredient | Composition (wt. %) |
|---|---|
| Humectant (e.g., sorbitol) | 10-20 |
| Sodium carboxymethyl cellulose | 0.1-1 |
| Glycerin | 20-30 |
| Potassium nitrate | 1-10 |
| Sodium saccharin | 0.1-1 |
| Sodium fluoride | 0.1-1 |
| Polyethylene glycol (peg 600) | 1-5 |
| Water | 10-20 |
| Synthetic high cleaning silica | 5-15 |
| Synthetic silica abrasive | 5-15 |
| Synthetic thickening silica | 1-5 |
| Flavor | 1-2 |
| 95% Sodium lauryl sulfate, granules | 1-5 |
| Dye | 0.001-0.01 |
| Cocamidopropyl betaine | 1-2 |
| Calcium pyrophosphate | 1-5 |
| Xantham gum | 0.1-1 |
| 50% KOH Caustic potash | 0.1-1 |
| Total | 100.000 |

### Example 4

Various compositions were prepared to analyze the effect of calcium pyrophosphate or tin oxide on fluoride stability. Results are reported in Table 4. The control sample utilized titanium dioxide.

TABLE 4

| | Initial | Fluoride | Fluoride | Fluoride |
|---|---|---|---|---|
| | Fluoride | at 4 weeks | at 8 weeks | at 13 weeks |
| | (ppm) | (ppm) | (ppm) | (ppm) |
| Control | 1075 | 1050 | 1080 | 1060 |
| 1% Calcium | 1020 | 1020 | 1030 | 1010 |
| pyrophosphate | | | | |
| 3% Calcium | 1031 | 992 | 1010 | 1000 |
| pyrophosphate | | | | |
| 5% Calcium | 993 | 967 | 971 | 969 |
| pyrophosphate | | | | |
| 0.5% Tin Oxide | 1180 | 1140 | 1070 | 1070 |
| 1% Tin Oxide | 1160 | 1120 | 1070 | 1040 |

### Example 5

Oral care compositions were prepared. Control compositions utilizing 0.5-1% of TiO₂ showed excellent opacity and whiteness. However, when TiO₂ was not added to the formula, the composition became milky white or appeared as a translucent gel material. When this composition was striped with other colors, such as blue or green, the stripe definition was poor and stripes were not present or faded with time in compositions having either 33%:34%:33%, 50%:50%, or 20%:80% stripes. (See FIGS. 1 to 3). Surprisingly, when the TiO₂ free striped compositions were formulated with 3% calcium pyrophosphate, stripping definition was significantly improved in compositions having either 33%:34%:33%, 50%:50%, or 20%:80% stripes. (See FIGS. 4 to 6).

### Example 6

Three exemplary oral care compositions having at least one opaque phase and at least one translucent phase were prepared in accordance with aspects of the invention. The oral care compositions were prepared to have the at least one opaque phase and at least one translucent phase in the form of stripes.

The first oral care composition comprised 34 wt. % of an opaque phase, 33 wt. % of a first translucent phase having a first color, and 33 wt. % of a second translucent phase having a second color. The second oral care composition comprised 80 wt. % of an opaque phase and 20 wt. % of a translucent phase having a color. The third oral care composition comprised 50 wt. % of an opaque phase and 50 wt. % of a translucent phase having a color. For all three oral care compositions, the opaque phases contained 3 wt. % of calcium pyrophosphate and the translucent phases contained 2 wt. % of calcium pyrophosphate, based on the total weight of the oral care composition.

The oral care compositions were packaged in respective toothpaste tubes and stored for a period of time. Each of the respective toothpaste tubes was squeezed to expel a portion of the oral care composition contained therein, thereby producing a sample of each exemplary oral care composition. The expelled oral care compositions were then visually evaluated. All of the oral care compositions had opaque phases and translucent phases that remained separate and in contact. Additionally, the interface between each of the phases was substantially uninterrupted between the end points of the sample, which is highly desirable.

While the present invention has been described with reference to several embodiments, which embodiments have been set forth in considerable detail for the purposes of making a complete disclosure of the invention, such embodiments are merely exemplary and are not intended to be limiting or represent an exhaustive enumeration of all aspects of the invention. The scope of the invention is to be determined from the claims appended hereto. Further, it will be apparent to those of skill in the art that numerous changes may be made in such details without departing from the spirit and the principles of the invention.

1-64. (canceled)

65. An oral care composition comprising:
from about 40 wt. % to about 75 wt. % of a humectant;
from about 5 wt. % to about 25 wt. % of an abrasive system;
from about 0.3 wt. % to about 1 wt. % of a thickening system; and
particles having a refractive index of from about 1.0 to about 2.5;
wherein the composition is substantially free of a titanium containing material, and all weight percentages are based on the total weight of the oral care composition.

66. The oral care composition according to claim 65, wherein the particles are selected from: a zinc compound; a calcium compound; a stannous compound; and a combination of two or more thereof.

67. The oral care composition according to claim 65, wherein the particles are selected from: zinc oxide; calcium pyrophosphate; dicalcium phosphate dihydrate; calcium carbonate; stannic oxide; and a combination of two or more thereof.

68. The oral care composition according to claim 65, wherein the particles comprise calcium pyrophosphate.

69. The oral care composition according to claim 65, wherein the particles have a refractive index from about 1.5 to about 2.0.

70. The oral care composition according to claim 65, wherein the particles are present in an amount of from about 0.1 wt. % to about 5 wt. %, based on the total weight of the oral care composition.

71. The oral care composition according to claim 65, wherein the oral care composition is substantially free of titanium containing materials.

72. The oral care composition according to claim 65, wherein the abrasive system comprises a silica having an average particle size of from about 3 microns to about 12 microns.

73. The oral care composition according to claim 65, wherein the composition has a Stripe Quality Index (SQI) score of greater than about 1.

74. An oral care composition comprising:
glycerin, sorbitol, or combinations thereof present in an amount from about 40% to about 75%, by weight of the oral care composition;
water present in an amount from about 5% to about 20%, by weight of the oral care composition;
silica abrasive present in an amount from about 5% to about 15% by weight of the oral care composition;
a thickening agent present in an amount from about 0.3% to about 1% by weight of the oral care composition; and
calcium pyrophosphate present in an amount from about 2% to about 5%, by weight of the oral care composition;
wherein the oral care oral care composition comprises a plurality of phases, wherein at least one phase is opaque, and wherein the oral care composition is substantially free of titanium dioxide.

75. The oral care composition according to claim 74, wherein the plurality of phases are in a form of strips.

76. The oral care composition according to claim 74, wherein adjacent phases of the plurality of oral care phases are in contact along an interface.

77. The oral care composition according to claim 74, wherein the thickening agent is selected from carboxyvinyl polymers, carrageenan, xanthan gum, hydroxyethyl cellulose and water-soluble salts of cellulose ethers, such as sodium carboxymethyl cellulose and sodium carboxymethyl hydroxyethyl cellulose, and a combination of two or more thereof.

78. The oral care composition according to claim 74, wherein the silica abrasive comprises colloidal particles having an average particle size of from about 7 microns to about 10 microns.

79. The oral care composition according to claim 74, wherein the composition is substantially free of titanium dioxide.

80. An oral care composition comprising:
glycerin, sorbitol, or combinations thereof present in an amount from about 40% to about 75%, by weight of the oral care composition;
water present in an amount from about 5% to about 20%, by weight of the oral care composition;
silica abrasive present in an amount from about 5% to about 15% by weight of the oral care composition;
a thickening agent present in an amount from about 0.3% to about 1% by weight of the oral care composition; and
tin oxide present in an amount from about 0.5% to about 2%, by weight of the oral care composition;
wherein the composition is substantially free of a titanium containing material.

81. The oral care composition according to claim 65, wherein the particles comprise tin oxide.

82. The oral care composition according to claim 65, wherein the particles comprise zinc oxide.

83. A method of treating, preventing or ameliorating a symptom associated with a disease, disorder or condition of the oral cavity, comprising applying an oral care composition according to claim 65, to an oral cavity surface.

84. The method according to claim 83, wherein the disease, disorder or condition of the oral cavity is selected from: gingivitis; periodontitis; excessive plaque and/or tartar build-up; caries; tooth decay; stained teeth; halitosis; erosion; sensitivity; inflammation; and a combination of two or more thereof.
